(19) United States
(12) Patent Application Publication
(10) Pub. No.: US 2026/0216343 A1
(43) Pub. Date: Jul. 30, 2026

(54) CONJUGATE, MICELLE AND ANTICANCER AGENT

(71) Applicant: TOHOKU UNIVERSITY, Sendai-shi, Miyagi (JP)

(72) Inventors: Masayuki YAMAMOTO, Sendai-shi (JP); Mikiko SUZUKI, Sendai-shi (JP); Kanjiro MIYATA, Tokyo (JP); Mitsuru NAITO, Tokyo (JP)

(21) Appl. No.: 19/148,904

(22) PCT Filed: Jan. 22, 2024
(86) PCT No.: PCT/JP2024/001650

§ 371 Date: Jul. 17, 2025

(30) Foreign Application Priority Data
Jan. 23, 2023 (JP) 2023-008006

(51) Int. Cl. A61K 47/54; A61K 47/60; A61K 47/65; A61K 47/69; A61P 35/00

(52) U.S. Cl. CPC A61K 47/545; A61K 47/60; A61K 47/65; A61K 47/6907; A61P 35/00

(57) ABSTRACT

A conjugate having a block copolymer having a PEG chain block and a polypeptide chain block in which one molecule or more of a compound (1) represented by the following general formula (1) is bonded to an amino acid side chain constituting the polypeptide chain block with a covalent bond through the oxygen atom of the hydroxy group of the compound (1). In the general formula (1), L¹ is a single bond or an alkylene group having one to five carbon atoms. Here, any methylene group in L¹ may be substituted with a carbonyl group or an ether bond as long as the oxygen atoms are not adjacent to each other. X¹ and X² are each independently a hydrogen atom or a halogen atom.

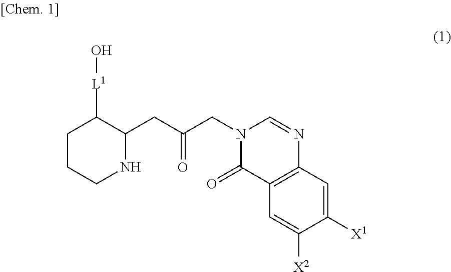

### CROSS-REFERENCE TO RELATED APPLICATIONS

This application is the U.S. National Stage entry of International Application No. PCT/JP2024/001650, filed on Jan. 22, 2024, which, in turn, claims priority to JP Patent Application No. 2023-008006, filed on Jan. 23, 2023, both of which are hereby incorporated herein by reference in their entireties for all purposes.

### TECHNICAL FIELD

The present invention relates to a conjugate containing a halofuginone compound, a micelle, and an anticancer agent.

### BACKGROUND ART

The Kelch-like ECH-associated protein 1 (Keap1)-NF-E2-related factor 2 (Nrf2) system is believed to contribute to cellular defense against endogenous or exogenous oxidative stress or xenobiotic stress. Keap1 is an adaptor protein rich in cysteine thiols, connects Nrf2 and Cullin-3 (Cul3), forms a ubiquitin E3 ligase complex and functions as a stress sensor of transcription factor Nrf2. In the cytoplasm in the resting state, Nrf2 is polyubiquitinated by a Keap1-Cul3 ubiquitin E3 ligase complex and is rapidly degraded in a proteasome-dependent manner. Here, cellular dysfunction due to exposure to electrophilic stress or the like deactivates the E3 ligase function of Keap1, and Nrf2 escapes from proteasomal degradation. Nrf2 forms heterodimers with small MAF Proteins (sMAF) in the nucleus, binds to CNC-sMAF binding elements (CsMBEs) and activates a group of cytoprotective genes. Standard target genes of Nrf2 include reactive oxygen species (ROS)-removing antioxidative enzymes such as NADPH: quinone oxidoreductase 1 (encoded by Nqo1), glutamate-cysteine ligase catalytic subunit and modifier subunit (Gclc and Gclm), heme oxygenase 1 (Hmox1), and peroxiredoxin 1 (Prdx1).

Cancer Genome Atlas (TCGA) includes data created by the newest sequence analysis method and helps identifying many pan-cancer somatic mutations which use various signal transduction pathways for driving and maintaining tumor development. The Keap1-Nrf2 pathway is one of such pathways frequently activated in cancer cells. Somatic mutations in Keap1 and Nrf2 genes are frequently observed in various types of cancer. Mutations that activate the Keap1-Nrf2 center are observed in about 30% of esophageal cancer cases, 34% of lung squamous carcinoma cases, and 19% of hepatocellular carcinoma cases.

Such mutations lead to abnormal production of Nrf2 in tumors. Activation of Nrf2 in host cells helps relieving many chemical stresses through coordinate activation of various genes encoding antioxidative stress response enzymes/proteins and the phase II metabolism system, but constructive accumulation of Nrf2 in cancer cells confers cancer cells with resistance to chemotherapies and radiotherapies and promotes proliferation of malignant tumors. It has been suggested that such constant accumulation of Nrf2 is induced when exclusive mutations are introduced in various regions in Nrf2, Keap1, or Cul3. In Nrf2, the DLG and ETGE motifs of the Neh2 degron are exceptionally responsible for the somatic mutations. The DLG and ETGE regions are extremely important for the Keap1-Nrf2 interaction. In Keap1 and Cul3, loss-of-functions mutations are found throughout the coding regions. In fact, the prognosis for patients having these mutations is poor, and the mortality rate also increases. For example, the average overall survival of non-small-cell lung cancer (NSCLC) patients with abnormal production of Nrf2 is as short as 11.2 months, while that of patients without the mutation is 36.8 months. Nrf2-activating mutations are more pertinent in the context of lung tumors, because 30% of all the NSCLC cases have a mutation in Keap1 and because 20% of all the KRAS-driven lung adenocarcinoma (LUAD) cases have a co-mutation in Keap1 or Nrf2. Therefore, a clinically highly relevant Keap1^{FB/FB}:Kras^{G12D} lung adenocarcinoma model system is sometimes used in researches.

Furthermore, because no approved therapeutic method is available for Nrf2-activated cancers, finding an effective therapeutic method for treating patients having such a tumor is an important unmet clinical need. Based on the findings and the reports, the present inventors have considered that use of an Nrf2 inhibitor would be a reasonable approach to such an Nrf2-addiction/-activated cancer. Thus, using high throughput screening by luciferase (Luc) assay of the chemical substance library of Faculty of Pharmaceutical Sciences of Tohoku University, halofuginone (HF) has been identified previously as a potent Nrf2 inhibitor, and its role as an anticancer chemical sensitizer has been preclinically demonstrated using Nrf2-activated cancer xenograft mice (NPL 1). The HF-induced Nrf2 accumulation was decreased through amino acid starvation response. HF inhibits prolyl-tRNA synthetase (PRS), and as a result, uncharged tRNAs accumulate in the cells. As a result, amino acid starvation response is caused, and the protein translation via the GCN2-eIF2 pathway is generally suppressed. In the preceding experiment, the present inventors have succeeded in regression of an Nrf2-activated tumor using HF, but the body weights of the mice reduced significantly when HF was used in combination with cisplatin, and toxicity in the mice was observed (NPL 1).

### CITATION LIST

### Non Patent Literature

NPL 1: Tsuchida K, Tsujita T, Hayashi M, Ojima A, Keleku-Lukwete N, Katsuoka F, Otsuki A, Kikuchi H, Oshima Y, Suzuki M, Yamamoto M. “Halofuginone enhances the chemo-sensitivity of cancer cells by suppressing NRF2 accumulation.” Free Radic Biol Med. 2017 February; 103:236-247.

### SUMMARY OF INVENTION

### Technical Problem

The experiment described in NPL 1 was conducted using nude mice having a KYSE70 xenograft without T cell immunity, and thus the present inventors have decided to use an advanced model system. Thus, a Keap1^{FB/FB}:Kras^{G12D} mouse model which is similar to Nrf2-activated lung adenocarcinoma due to Kras was used for examination.

Moreover, according to the preceding research by the present inventors, Nrf2 inhibition in a tumor is believed to be a reasonable approach to treatment of an Nrf2-activated cancer, but activation of Nrf2 in host immune cells has also been suggested to suppress progress and metastasis of a tumor. Accordingly, it is believed that tumor-specific Nrf2 suppression, if possible, may lead to superior suppression of a tumor.

Thus, to achieve tumor specificity and induce therapeutic reaction with less side effects, the present inventors have intensively studied to deliver HF as nanomedicine and to passively target an Nrf2-activated cancer, and the invention has been thus completed.

The invention provides a conjugate, a micelle, and an anticancer agent which reduce the systemic toxicity of halofuginone and which have a tumor suppression action.

### Solution to Problem

The invention has the following aspects.
[1]A conjugate having a block copolymer having a PEG chain block and a polypeptide chain block in which one molecule or more of a compound (1) represented by the general formula (1) described below is bonded to an amino acid side chain constituting the polypeptide chain block with a covalent bond through the oxygen atom of the hydroxy group of the compound (1).
[2] The conjugate according to [1] in which the covalent bond is an ester bond or a urethane bond.
[3] The conjugate according to [1] in which one to 100 molecules of the compound (1) are bonded to one molecule of the block copolymer.
[4] The conjugate according to any one of [1] to [3] in which the polypeptide chain block is a polyaspartic acid chain or a polyglutamic acid chain.
[5] The conjugate according to any one of [1] to [4] in which the polymerization degree of the polypeptide chain block is 10 to 100.
[6] The conjugate according to any one of [1] to [5] in which the polymerization degree of the PEG chain block is 50 to 900.
[7] The conjugate according to any one of [1] to [6] in which a side chain of the polypeptide chain block forms a pharmaceutically acceptable salt.
[8] The conjugate according to any one of [1] to [7] which is represented by the general formula (2) described below.
[9] The conjugate according to any one of [1] to [8] which is represented by the general formula (3) described below.
[10]A micelle formed by association of a plurality of the conjugates according to any one of [1] to [9].
[11] The micelle according to [10] having a hydrodynamic diameter at 25° C. of 30 to 50 nm.
[12] An anticancer agent containing the conjugate according to any one of [1] to [9] or the micelle according to the item [10] or [11] as an active ingredient.

### Advantageous Effects of Invention

According to the invention, a conjugate, a micelle, and an anticancer agent which reduce the systemic toxicity of halofuginone and which have a tumor suppression action can be provided.

When the conjugate or the micelle according to the invention is used as nanomedicine, accumulation of halofuginone and analogues thereof in a healthy organ is prevented, and the possibility of a side effect is reduced. As a result, the therapeutic range of halofuginone and the analogues thereof can be extended.

As shown as an example in the Examples described below, a new polymer micelle in which halofuginone is incorporated in a poly(ethylene glycol)-poly(peptide) block copolymer through an ester bond (hereinafter sometimes referred to as HFm) was developed, and the efficiency of removing Nrf2-activated lung adenocarcinoma in a mouse model was evaluated. As a result, it was found that free halofuginone which was not in the micelle form (hereinafter sometimes simply referred to as HF) and HFm both suppressed an Nrf2-activated lung tumor with significance in mice and that the acute systemic toxicity of HF administration was reduced with HFm administration. The results clearly show that HFm is a potent Nrf2 inhibitor with low toxicity, and HFm may play an important role in clinical cases in the future.

Here, the Keap1-Nrf2 system is a master regulator of cellular response to oxidative stress and xenobiotic substances. Constant activation of Nrf2 is frequently observed in various types of cancer. Excessive activation of Nrf2 causes reprogramming of the metabolism of cancer cells, supports an increase in the energy demand required for rapid proliferation and confers with high resistance to radio-/chemotherapies with an anticancer agent. Accordingly, an Nrf2 inhibitor has emerged as an attractive therapy strategy against such acquired resistance in an Nrf2-activated tumor.

In NPL 1, free halofuginone (HF) has been identified as a promising Nrf2 inhibitor. The present inventors have conducted preclinical trials of HF and have found that HF significantly decreases the survival rate of cancer cells but also causes serious suppression of hematopoietic immune cells in a dose-dependent manner. Thus, to overcome the toxicity, the present inventors have decided to employ nanomedicine approach of HF. As shown as examples in the Examples described below, it has been found that the systemic toxicity of HF can be relieved considerably while the tumor suppression action of HF is maintained when HF is encapsuled in a polymer micelle. It has been elucidated by LC-MS/MS analysis that the relief of the side effect is a result of slow and continuous release of HF from the core of HFm. The results support the claim that HFm may be able to act against an Nrf2-activated cancer in clinical cases.

### BRIEF DESCRIPTION OF DRAWINGS

FIG. 1A The chemical structure of halofuginone (HF)

FIG. 1B A schematic view of the HF/saline treatment protocol of Keap1 KO-mCherry cells and WT-GFP cells.

FIG. 1C Fluorescence intensities of Keap1 KO-mCherry cells and WT-GFP cells 48 hours after introducing HF at different concentrations. The fluorescence levels were normalized by the fluorescence levels of the saline-treated controls (n=3). ***P≤0.001 (Student t-test).

FIG. 1D Typical images of the cells 48 hours after saline or HF (50 nM) treatment. Scale—200 μm.

FIG. 1E The Nrf2 protein levels in Keap1 KO-mCherry cells and WT-GFP cells after HF treatment (50 nM) at indicated times.

FIG. 1F Quantification of the Nrf2 protein levels of the whole cell lysates of Keap1 KO-mCherry and WT-GFP cells exposed to 50 nM HF for the indicated times (n=3). At each point in time, the level is compared with that of the control (0 hour) of the same group (Keap1 KO or WT). *P≤0.05, **P≤0.01 (non-parametric Kruskal-Wallis test). The Nrf2 protein was normalized by the α-tubulin level. The data shown in the graph are average values±SD.

FIG. 2A The experiment strategy for determining the maximum tolerated dose (MTD) of HF.

FIG. 2B The changes in body weight after intravenous injection of HF (n=7). The mice treated with 1 mg/kg of HF (broken line) were moribund and were thus euthanized before the completion of experiment. Student t-test, *P≤0.05 day 1 vs. day 4, t one out of eight mice died at 1 mg/kg.

FIG. 2C The leukocyte counts, the erythrocyte counts, and the platelet counts in the peripheral blood of the mice with different HF treatment amounts. *P≤0.05, ns=not significant (non-parametric Kruskal-Wallis test).

FIG. 2D The spleen weights, the total spleen cell counts, and the erythroblast counts (Ter119⁺CD71⁺) after gradually increasing HF treatment. The cells were isolated from the spleens of mice. *P≤0.05, **P≤0.01, ***P≤0.001 ns=not significant (non-parametric Kruskal-Wallis test).

FIG. 2E The total cells, the precursor cells (c-Kit⁺), and the B (B220⁺) cells obtained from the bone marrows of mice treated with HF at different concentrations. *P≤0.05, **P≤0.01, ns=not significant (non-parametric Kruskal-Wallis test).

FIG. 2F The thymus weights, the total thymus cell counts, and the CD4 single-positive (CD4⁺CD8⁻) and CD8 single-positive (CD4⁻CD8⁺) cell counts after HF treatment. The cells were isolated from the thymi. *P≤0.05, ns=not significant (non-parametric Kruskal-Wallis test). The data shown in the graph are average values±SD.

FIG. 3A The chemical structures of PEG-PAsp(HF) and a micelle thereof.

FIG. 3B A ¹H NMR spectrum of PEG-PAsp(HF). “a” and “b” correspond to benzyl protons of HF and protons of the polymer, respectively.

FIG. 3C A size distribution histogram of HFm measured by dynamic light scattering.

FIG. 3D The experiment design of LC-MS/MS analysis where saline, HF, or HFm (both at 0.75 mg/kg) was injected once to WT mice (eight to 12 weeks old). The spleens, the livers, and the lungs were collected 0, 24, 48, 96, and 144 hours after the intravenous injection.

FIG. 3E The accumulated HF amounts in the spleen, liver, and lung tissues at different points in time after HF and HFm (0.75 mg/kg) treatment. *P≤0.05, ns=not significant (non-parametric Mann-Whitney test). The bars showing the measurement values at each time point show the results of saline, HF, and HFm, respectively, from the left.

FIG. 4A The experiment strategy for examining the tumor suppression effects of HF and HFm. Adeno-Cre virus was intranasally administered to Keap1^{FB/FB}:Kras^{G12D} mice (eight to 12 weeks old) to induce Nrf2-activated lung adenocarcinoma. The tumors were grown for 17 weeks and then treated with saline, HF, or HFm every two days four times in total.

FIG. 4B The lung weights of mice after saline, HF, or HFm treatment; non-parametric Kruskal-Wallis test, **P≤0.01, ns=not significant.

FIG. 4C Typical lung slices stained with hematoxylin-eosin (HE) (top and middle) and elastica-Masson (bottom) of cancer-bearing mice treated with saline, HF, or HFm. Scale—5 mm (top) and 50 μm (middle and bottom).

FIG. 4D The tumor areas of the saline-, HF-, or HFm-treated mice were measured using the HE-stained lung sections. The dark stained parts in the HE-stained lung sections show the tumors. *P≤0.05, ns=not significant (non-parametric Kruskal-Wallis test).

FIG. 4E The tumor grades were determined based on the elastica-Masson-stained lung sections. There are two types of lung tumors, namely lepidic lesion and papillary lesion. When elastin is observed in the section (the arrows with “x” in FIG. 4C), the tumor is classified as lepidic tumor (Grade I) and speculated to grow in situ. When such a pattern is not observed and when elastin appears as dots (the arrows with “y” in FIG. 4C), the tumor is classified as papillary tumor (Grade II). When most of the part is composed of a solid sheet and cannot be classified as the lepidic form or the papillary form, the tumor is called solid tumor (Grade III). Here, in HF/HFm-treated mice, the solid tumor (Grade III) disappeared completely. **P≤0.01, ns=not significant (non-parametric Kruskal-Wallis test). The data shown in the graph are average values±SD.

FIG. 5A The leukocyte counts, the erythrocyte counts, and the hematocrit levels of the peripheral blood of cancer-bearing mice (Keap1^{FB/FB}:Kras^{G12D}) treated with saline, HF (0.75 mg/kg), or HFm (0.75 mg/kg). *P≤0.05, ns=not significant (non-parametric Kruskal-Wallis test).

FIG. 5B The spleen weights, the total spleen cell counts, and the erythroblasts (Ter119⁺CD71⁺) after saline, HF, and HFm treatment. The cells were isolated from the spleens of mice. *P≤0.05, **P≤0.01, ***P≤0.001, ns=not significant (one-way analysis of variance and Tukey's multiple comparison test for the spleen weights, and non-parametric Kruskal-Wallis test for the total spleen cell counts and the erythroblasts).

FIG. 5C The thymus weights, the total thymus cells, the double-negative cells (CD4⁻CD8⁻), the double-positive cells (CD4⁺CD8⁺), the CD4 single-positive cells (CD4⁺CD8⁻), and the CD8 single-positive cells (CD4⁻CD8⁺) after saline, HF, and HFm treatment. The cells were isolated from the thymi. *P≤0.05, **P≤0.01, ***P≤0.001, ****P≤0.0001, ns=not significant (one-way analysis of variance and Tukey's multiple comparison test for the thymus weights, and non-parametric Kruskal-Wallis test for the thymus cell counts).

FIG. 5D The total cells, the precursor cells (c-Kit⁺), the B cells (B220⁺), the macrophages (CD11b⁺F4/80⁺), and the neutrophils (CD11b⁺Ly6G⁺) isolated from cancer-bearing mice (Keap1^{FB/FB}:Kras^{G12D}) treated with saline, HF, or HFm. Serious depletion of hematopoietic cells and immune cells was observed by HF treatment. The cell death was inhibited when HFm was used. *P≤0.05, **P≤0.01, ***P≤0.001, ns=not significant (non-parametric Kruskal-Wallis test). The data shown in the graph are average values±SD.

FIG. 6A A schematic view of the protocol according to LC-MS/MS analysis of different organs of cancer-bearing mice (Keap1^{FB/FB}:Kras^{G12D}). Saline, HF (0.75 mg/kg), or HFm (0.75 mg/kg) was injected to cancer-bearing mice every two days four times in total. Samples were collected on the eighth day.

FIG. 6B The HF levels in the lung, spleen, and liver samples of cancer-bearing mice after saline, HF (0.75 mg/kg), or HFm (0.75 mg/kg) treatment. The accumulated HF amount with the HFm administration was lower with significance than that with the free HF treatment. *P≤0.05, **P≤0.01, ns=not significant (non-parametric Mann-Whitney test). The data shown in the graph are average values±SD.

FIG. 7A The therapy protocol conducted during the experiment. Adeno-Cre virus was intranasally administered to Keap1^{FB/FB}:Kras^{G12D} mice (eight to 12 weeks old), and the tumors were grown for 17 weeks. Then, the mice were treated with saline, HF (0.5 mg/kg), or HFm (0.25 mg/kg) every two days four times in total.

FIG. 7B The lung weights of the mice after saline, HF, or HFm treatment. **P≤0.01, ns=not significant (non-parametric Kruskal-Wallis test).

FIG. 7C Typical lung slices stained with hematoxylin-eosin (HE) (top and middle) and elastica-Masson (bottom) of cancer-bearing mice treated with saline, HF, or HFm. Scale—5 mm (top) and 50 μm (middle and bottom).

FIG. 7D The tumor areas of the saline-, HF-, or HFm-treated mice were measured using the HE-stained lung sections. The dark stained parts in the HE-stained lung sections show the tumors. *P≤0.05, **P≤0.01, ns=not significant (non-parametric Kruskal-Wallis test).

FIG. 7E The tumor grades were determined based on the elastica-Masson-stained lung sections. The numbers of lepidic tumors (Grade I) and solid tumors (Grade III) were lower in the HF- and HFm-treated mice with significance than in the saline-treated mice. *P≤0.05, ns=not significant (non-parametric Kruskal-Wallis test). The solid tumors did not disappear completely as in the cases with treatment with high doses of HF or HFm.

FIG. 7F The body weights were observed every two days and changed in the similar manner in the three groups.

FIG. 7G The leukocytes and the erythrocytes in the peripheral blood of the saline-, HF-, or HFm-treated mice. Here, a significant decrease in the leukocytes is observed in the HF-treated mice, but such a side effect is not observed with HFm injection. *p≤0.05, ***p≤0.001, ns=not significant (non-parametric Kruskal-Wallis test). The data shown in the graph are average values±SD.

### DESCRIPTION OF EMBODIMENTS

The invention will be explained in detail below. The invention, however, is not limited to the following embodiments. The terms in the present description are defined below.

In the present description and the claims, the lower limit value and the upper limit value of a numerical range with “to” are included in the numerical range. That is, when a numerical range is “1 to 10”, for example, the numerical range means the numerical range including the lower limit value 1 and the upper limit value 10.

### <<Halofuginone Compound>>

The halofuginone compound contained in the conjugate of the invention is the compound (1) represented by the following general formula (1).

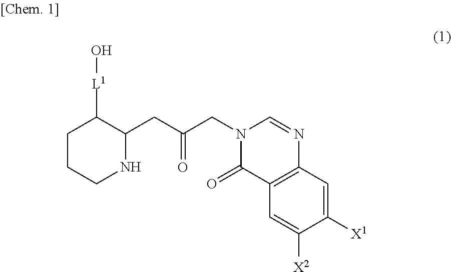

In the general formula (1), L¹ is a single bond or an alkylene group having one to five carbon atoms. Here, any methylene group in L¹ may be substituted with a carbonyl group (—C(═O)—) or an ether bond (—O—) as long as the oxygen atoms are not adjacent to each other. That is, L¹ may contain —O—, —C(═O)—O—, or —O—C(═O)—, for example. X¹ and X² are each independently a hydrogen atom or a halogen atom.

In the general formula (1), L¹ is a single bond or an alkylene group having one to five carbon atoms, and any methylene group in L¹ may be a carbonyl group or an ether bond. To enhance the tumor suppression action, L¹ is preferably a single bond.

In the general formula (1), X¹ and X² are each a hydrogen atom or a halogen atom, and to reduce the systemic toxicity, X¹ and X² are each preferably a halogen atom, more preferably a chlorine atom or a bromine atom. Here, X¹ and X² may be the same or different.

In the general formula (1), to reduce the systemic toxicity, X¹ is preferably a bromine atom, and X² is preferably a chlorine atom.

The compound of the general formula (1) in which L¹ is a single bond and X¹ and X² are hydrogen atoms is also called febrifugine.

The compound of the general formula (1) in which L¹ is a single bond, X¹ is a bromine atom, and X² is a chlorine atom is halofuginone.

The compound (1) may form a pharmaceutically acceptable salt.

Examples of the pharmaceutically acceptable salt of the compound (1) include a salt with an inorganic acid and a salt with an organic acid. Examples of the salt with an inorganic acid include salts with hydrochloric acid, hydrobromic acid, hydroiodic acid, nitric acid, sulfuric acid, phosphoric acid, and the like. Examples of the salt with an organic acid include salts with acetic acid, oxalic acid, fumaric acid, maleic acid, succinic acid, malic acid, citric acid, tartaric acid, adipic acid, gluconic acid, glucoheptic acid, glucronic acid, terephthalic acid, methanesulfonic acid, alanine, lactic acid, hippuric acid, 1,2-ethanedisulfonic acid, isethionic acid, lactobionic acid, oleic acid, gallic acid, pamoic acid, polygalacturonic acid, stearic acid, tannic acid, trifluoromethanesulfonic acid, benzenesulfonic acid, p-toluenesulfonic acid, naphthalenesulfonic acid, and sulfosalicylic acid. Of these, the pharmaceutically acceptable salt of the compound (1) is preferably a salt with hydrobromic acid.

The halofuginone of this aspect is produced by a known method. For example, the halofuginone of this aspect can be acquired from Carbosynth Limited as a salt with hydrobromic acid.

The halofuginone compound of this aspect can be synthesized, for example, from halofuginone or febrifugine as a starting material. Moreover, a halofuginone compound other than halofuginone and febrifugine can be synthesized by modifying a part of the process of synthesizing halofuginone or febrifugine from various materials by a known method based on the chemical knowledge apparent to one skilled in the art.

Here, the pharmaceutical action of halofuginone (HF) is described in NPL 1 and is as follows although the following description includes other findings and speculation.

The Keap1-Nrf2 pathway is one of pathways frequently activated in cancer cells. Somatic mutations in Keap1 and Nrf2 genes are frequently observed in various types of cancer. Such mutations lead to abnormal production of Nrf2 in tumors. Activation of Nrf2 in host cells helps relieving many chemical stresses through coordinate activation of various genes encoding antioxidative stress response enzymes/proteins and the phase II metabolism system, but constructive accumulation of Nrf2 in cancer cells confers cancer cells with resistance to chemotherapies and radiotherapies and promotes proliferation of malignant tumors. HF is a potent Nrf2 inhibitor and inhibits prolyl-tRNA synthetase (PRS), and as a result, uncharged tRNAs accumulate in the cells. As a result, amino acid starvation response is caused, the protein translation via the GCN2-eIF2 pathway is generally suppressed, and a decrease in Nrf2 accumulation is induced. Thus, HF can significantly decrease the survival rate of cancer cells.

In this regard, however, the invention is not restricted by the above mechanism.

The general formula (1) represents halofuginone and a compound having a similar chemical structure to that of halofuginone and thus may be referred to as a halofuginone compound. It is supported from the similar chemical structure that the halofuginone compound exhibits the same pharmaceutical properties to those of halofuginone.

### <<Conjugate>>

A first aspect of the invention is a conjugate having a block copolymer having a PEG chain block and a polypeptide chain block in which one molecule or more of a halofuginone compound represented by the formula (1) is bonded to an amino acid side chain constituting the polypeptide chain block with a covalent bond through the oxygen atom of the hydroxy group of the halofuginone compound.

A plurality of the conjugates of this aspect can aggregate and form a micelle containing the halofuginone compound inside (HFm). It is believed that, because the halofuginone compound is relatively hydrophobic compared to the PEG chain block and the polypeptide chain block, HFm contains the hydrophobic halofuginone compound inside, and the PEG chain block mainly constitutes the shell of HFm. As shown in the Examples described below, HFm is easily delivered to cancer cells and releases the free halofuginone compound gradually in the cancer cells. As a result, the systemic toxicity of the halofuginone compound is reduced, and a tumor suppression action is exhibited.

### (PEG Chain Block)

The PEG chain block of this aspect generally has a PEG chain represented by the following general formula (P). H[—O—CH₂—CH₂]1—OH . . . (P)

In the general formula (P), 1 is an integer representing the average polymerization degree of the PEG chain. Here, the average polymerization degree of the PEG chain indicates the average repeat number of the oxyethylene moiety.

Because HFm is easily formed, the polymerization degree 1 of the PEG chain constituting the PEG chain block of this aspect is, for example, preferably 50 to 900, more preferably 100 to 500, further preferably 170 to 380, particularly preferably 220 to 330.

In the PEG chain block of this aspect, at least one of the hydroxy groups at both ends of the PEG chain is bonded to the N-terminus or the C-terminus of the polypeptide chain block with or without any linking group to form a block copolymer.

Here, the linking group can link the PEG chain block and the polypeptide chain block, and examples thereof include a single bond, a divalent organic group, a disulfide bond (—S—S—), and —R₂Si—O— (here, the two R's bonded to the silicon atom are a hydrogen atom, a methyl group, or an ethyl group). Examples of the divalent organic group include an alkylene group having one to six carbon atoms, —CH₂—N—, a group containing a triazole formed by click chemistry, and the like. Any methylene group constituting the alkylene group may be substituted with a carbonyl group or an ether bond as long as the oxygen atoms are not adjacent to each other, and more specifically, —O—, —C(═O)—O—, —O—C(═O)—, or the like may be contained.

The linking group may be a group derived from a polymerization initiator group at a terminus of the PEG chain block. Here, the polymerization initiator group refers to a group from which the polymerization of the polypeptide chain initiates and which can constitute a terminus of the polypeptide chain. A PEG chain block having such a polymerization initiator group (for example, an amino group or a carboxy group) at a terminus is sometimes referred to as a macro-initiator. That is, the linking group may be a linking group derived from the macro-initiator. When the macro-initiator has an amino group, the linking group can be an imino group.

### (Polypeptide Chain Block)

The polypeptide chain block of this aspect has a polypeptide chain in which amino acids are linearly polymerized with peptide bonds.

One kind or two or more kinds of amino acid (sometimes referred to as an amino acid unit in the present description) may constitute the polypeptide chain, but at least one amino acid having a side chain which can be bonded to the halofuginone compound with a covalent bond is included. Examples of the amino acid constituting the polypeptide chain include alanine, valine, leucine, isoleucine, glycine, serine, threonine, methionine, cysteine, phenylalanine, tyrosine, tryptophan, asparagine, glutamine, aspartic acid, glutamic acid, lysine, arginine, histidine, and proline.

The amino acid constituting the polypeptide chain may be in the L-form or the D-form. Moreover, the amino acid constituting the polypeptide chain may be a derivative or an analogue of an amino acid.

The amino acid constituting the polypeptide chain is preferably an amino acid having an acidic side chain or a basic side chain because the side chain easily forms a covalent bond with the halofuginone compound. Of these, because the covalent bond with the halofuginone compound is particularly easily formed, aspartic acid and glutamic acid are preferable, and aspartic acid is more preferable.

When the polypeptide chain has an aspartic acid unit, the aspartic acid unit content based on the total number of all the amino acid units constituting the polypeptide chain is preferably 10 to 100%, more preferably 50 to 100%, further preferably 80 to 100%, because the covalent bond with the halofuginone compound is easily formed. Here, the amino acid units other than the aspartic acid unit may be any units.

When the polypeptide chain has an aspartic acid unit, the glutamic acid unit content based on the total number of all the amino acid units constituting the polypeptide chain is preferably 10 to 100%, more preferably 50 to 100%, further preferably 80 to 100%, because the covalent bond with the halofuginone compound is easily formed. Here, the amino acid units other than the glutamic acid unit may be any units.

The polymerization degree of the polypeptide chain constituting the polypeptide chain block of this aspect is, for example, preferably 10 or more because HFm is easily formed, and the polymerization degree is preferably 10 to 100, more preferably 20 to 70, further preferably 25 to 55 to control the hydrodynamic diameter of the micelle in a range suitable for the drug delivery.

The order and the arrangement of the amino acid units constituting the polypeptide chain block of this aspect may be any order and any arrangement. In the polypeptide chain block of this aspect, miniblocks each formed with a same kind of amino acid unit may be included, or the amino acid units may be located randomly.

### (Block Copolymer)

The block copolymer of this aspect has one or more PEG chain blocks and one or more polypeptide chain blocks.

The numbers and the arrangement order of the blocks, seen from one terminus to the other terminus of the block copolymer, can be any numbers and any arrangement order. The blocks may be bonded with or without any linking group.

In the conjugate of this aspect, because HFm can be easily formed, the block copolymer preferably has one PEG chain block and one polypeptide chain block. In this case, the PEG chain block may be bonded to the N-terminus of the polypeptide chain block or to the C-terminus but is preferably bonded to the C-terminus.

The block copolymer of this aspect may have a constituent unit derived from a macro-initiator used during the synthesis. Here, the macro-initiator is a polymer that has a functional group which initiates the polymerization reaction of the amino acid monomer or a functional group from which the polymerization of the amino acid monomer initiates. The macro-initiator is preferably a macro-initiator having a PEG chain and having a functional group which reacts with the amino group or the carboxy group of the amino acid monomer at a terminus of the PEG chain. Examples of the functional group include an amino group, a carboxy group, and a thiol group.

An example of the macro-initiator is commercial α-methoxy-ω-aminopropyl-poly(ethylene glycol).

### (Covalent Bond)

In the conjugate of this aspect, the type of the covalent bond between the halofuginone compound and an amino acid side chain constituting the polypeptide chain block is not particularly limited. Examples of the covalent bond include an ester bond and a urethane bond. Of these, to form a covalent bond with an amino acid side chain, especially aspartic acid and glutamic acid side chains, an ester bond is preferable. An ester bond and a urethane bond can be hydrolyzed in the cells. Because the covalent bonds can be cleaved in the cells, free HF can be gradually released from the micelle (HFm).

The number of molecules of the halofuginone compound bonded to the conjugate of this aspect with the covalent bond, per one molecule of the block copolymer, is, for example, preferably one to 100, more preferably three to 50, further preferably six to 20.

When the number is the lower limit value of the range or more, the action as an anticancer agent is enhanced, and when the number is the upper limit value of the range or less, the synthesis of HFm is easy, and the stability of HFm is also increased.

The number of molecules of the halofuginone compound bonded to the conjugate of this aspect with the covalent bond, based on the total number of the amino acid units constituting the conjugate, is, for example, 10 to 60% and may be 20 to 50% or 30 to 40%.

When the number is the lower limit value of the range or more, the action as an anticancer agent is enhanced, and when the number is the upper limit value of the range or less, the synthesis of HFm is easy, and the stability of HFm is also increased.

The ester bond can be synthesized, for example, by dehydration condensation of the hydroxy group of the side chain of the amino acid unit and the hydroxy group of the halofuginone compound.

The urethane bond can be synthesized, for example, by linking the amino group of the side chain of the amino acid unit and the hydroxy group of the halofuginone compound with a carbonyl group. The urethane bond can be formed according to a known method. For example, the urethane bond can be formed by reacting the amino group of the side chain of the amino acid unit with a condensing agent and then reacting the hydroxy group of the halofuginone compound and the reaction product. An example of the condensing agent is carbonyldiimidazole.

A preferable embodiment of the conjugate of this aspect is a conjugate represented by the following general formula (2).

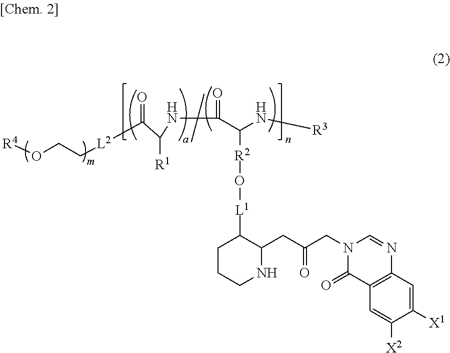

In the general formula (2), because the description of L¹, X¹, and X² is the same as the description for the general formula (1) above, overlapping description is not given.

m is an integer of 50 to 900 representing the average polymerization degree of the PEG chain. n is an integer of 10 or more representing the polymerization degree of the polypeptide chain. a is a number of 0≤a<1 that represents the proportion of the constituent unit based on an amino acid to which the compound (1) is not bonded. b is a number of 0<b≤1 that represents the proportion of the constituent unit based on an amino acid to which the compound (1) is bonded. Here, a+b=1.

R¹ is an amino acid side chain. R² represents an amino acid side chain but forms a covalent bond with the compound (1).

R³ is a hydrogen atom, a functional group, or an atomic group which constitutes the terminus of the polypeptide chain block. The functional group or the atomic group is not particularly restricted as long as the group does not prevent the formation of HFm or inhibit the function as an anticancer agent. A known one which modifies a terminus of a polypeptide chain can be applied, and an example thereof is a hydrogen atom.

R⁴ is a hydrogen atom, a functional group, or an atomic group which constitutes the terminus of the PEG chain block. The functional group or the atomic group is not particularly restricted as long as the group does not inhibit the function of HFm as an anticancer agent, and an example thereof is an alkyl group having one to three carbon atoms.

Because the description of L² in the general formula (2) is the same as the description of the linking group, overlapping description is not given here.

Because the description of m in the general formula (2) is the same as the description of the polymerization degree of the PEG chain block, overlapping description is not given here.

Because the description of n in the general formula (2) is the same as the description of the polymerization degree of the polypeptide chain block, overlapping description is not given here.

In the general formula (2), a is a number in the range 0≤a<1 and is preferably 0.4 to 0.9, more preferably 0.5 to 0.8, further preferably 0.6 to 0.7.

When a is the lower limit value of the range or more, the synthesis of HFm is easy, and the stability of HFm is also increased. When a is the upper limit value of the range or less, the action as an anticancer agent is enhanced.

In the general formula (2), b is a number in the range 0<b≤1 and is preferably 0.1 to 0.6, more preferably 0.2 to 0.5, further preferably 0.3 to 0.4.

When b is the lower limit value of the range or more, the action as an anticancer agent is enhanced, and when b is the upper limit value of the range or less, the synthesis of HFm is easy, and the stability of HFm is also increased.

Because the description of the PEG chain block and the polypeptide chain block contained in the general formula (2) is the same as the description given above, overlapping description is not given.

In the general formula (2), the oblique line between the amino acid unit having R¹ and the amino acid unit having R² shows an aspect in which the polypeptide chain block has the amino acid unit having R¹ and the amino acid unit having R² at proportions a and b, respectively. That is, the oblique line does not limit the arrangement of the amino acid unit having R¹ and the amino acid unit having R².

A more preferable embodiment of the conjugate of this aspect is a conjugate represented by the following general formula (3), for example.

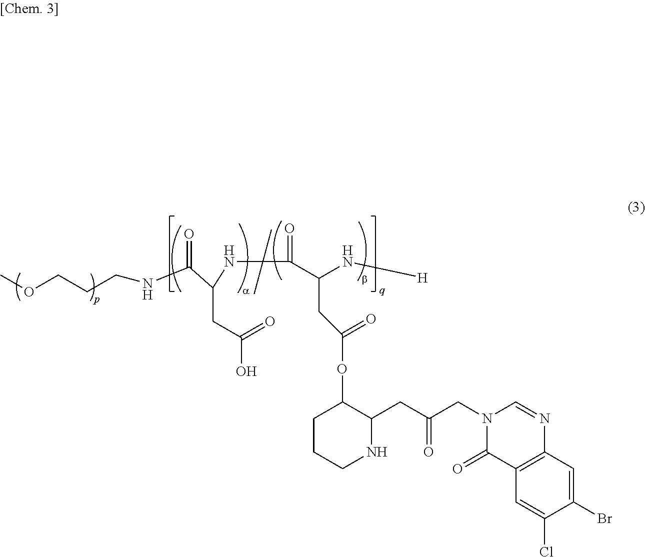

In the general formula (3), p is an integer of 170 to 380 representing the polymerization degree of the PEG chain. q is an integer of 25 to 55 representing the polymerization degree of the polypeptide chain. α is a number of 0.4≤α≤0.7 that represents the proportion of the constituent unit based on aspartic acid to which halofuginone is not bonded. B is a number of 0.3≤β≤0.6 that represents the proportion of the constituent unit based on aspartic acid to which halofuginone is bonded. Here, α+β=1.

The oblique line between the constituent unit based on the aspartic acid to which the halofuginone is not bonded and the constituent unit based on the aspartic acid to which the halofuginone is bonded is drawn to show an aspect in which the polypeptide chain block has the amino acid units at proportions a and β, respectively, and the oblique line does not limit the arrangement of the amino acid units.

The conjugate of this aspect can be obtained by, for example, any of the first to third production methods below.

The first production method has, for example, a step of reacting a block copolymer having a PEG chain block and a polypeptide chain block obtained by a known method with a halofuginone compound to obtain a conjugate.

The second production method has, for example, a step of reacting a macro-initiator having a PEG chain and an amino acid monomer or an amino acid unit precursor to obtain a block copolymer having a PEG chain block and a polypeptide chain block and a step of reacting the block copolymer and a halofuginone compound to obtain a conjugate.

The third production method has, for example, a step of reacting a macro-initiator having a PEG chain and an amino acid monomer or an amino acid unit precursor having a protective group at a side chain to polymerize the amino acid monomer or the amino acid unit precursor having the protective group, a step of removing the protective group in the obtained block copolymer, and a step of reacting the block copolymer and a halofuginone compound to obtain a conjugate.

An example of the amino acid unit precursor is α-amino acid-N-carboxyanhydride (NCA). NCA is a molecule in which the amino group and the carboxy group of an amino acid having a side chain form a ring through a carbonyl group (C═O). It is known that, when a base is caused to act on NCA, NCA is polymerized with cleavage of the ring to provide a polypeptide chain. The synthesis method of NCA is known, and a commercial product is also available.

### <<Micelle>>

A second aspect of the invention is a micelle formed by association of a plurality of the conjugates of the first aspect. The micelle contains a halofuginone compound. In the present description, a micelle containing a halofuginone compound is sometimes referred to as HFm.

In general, a micelle often means a small particle formed with one layer of molecular film, but the form of the micelle of this aspect is not limited thereto and may be another form. The structure of HFm may be a structure in which the PEG chain block mainly forms the shell of the micelle, the halofuginone compound is mainly included in the micelle, and the polypeptide chain block is mainly at the position connecting the inside of the micelle and the shell.

The number of molecules of the conjugate constituting HFm may be a number capable of forming a micelle, is, for example, four to 24 and may be eight to 20 or 12 to 16.

The number of molecules of the halofuginone compound contained in the micelle is not particularly restricted but is, for example, 40 to 2400 and may be 100 to 2000 or 150 to 1500 to enhance the function as an anticancer agent and in view of the stability of the micelle.

The number of molecules of the halofuginone compound contained in the micelle, based on the total number of the amino acid units contained in the conjugate constituting the micelle, is, for example, 10 to 60% and may be 20 to 50% or 30 to 40%.

Because the drug delivery to cancer cells in the body is easy, the hydrodynamic diameter of HFm at 25° C. is preferably 50 nm or less, preferably 30 to 50 nm, more preferably 30 to 45 nm, further preferably 30 to 40 nm.

Here, the hydrodynamic diameter of HFm is a value measured at 25° C. using a laser diffraction/scattering measurement device by dynamic light scattering (DLS). By dynamic light scattering, for example, the average particle size d (hydrodynamic diameter) and the polydispersity index are determined by the cumulant method from the autocorrelation function determined by the photon correlation method, and the particle size distribution is determined by the histogram method.

A preferable measurement method of the hydrodynamic diameter is explained next. The hydrodynamic diameter can be measured, for example, with a Zetasizer (manufactured by Malvern). Regarding the size, the diffusion of the particles moving by the Brownian motion is measured, and the measurement results are converted to the particle size and the particle size distribution using the Stokes-Einstein equation. According to the need, the shape of the micelle is evaluated using a transmission electron microscope. Here, the Z-average particle size is the data of a particle dispersion or the like which are measured by dynamic light scattering and analyzed using the cumulant analysis method. In the cumulant analysis, the average value and the polydispersity index (PDI) of the particle size are obtained. In this aspect, the average particle size is defined as the Z-average particle size. In the strict sense, the operation of fitting a polynomial to the logarithm of the G1 correlation function obtained by measurement is called the cumulant analysis, and the constant b in the equation:

LN(G1)=a+bt+ct2+dt3+et4+ . . . is called the second cumulant or the Z-average diffusion coefficient. The value obtained by converting the value of the Z-average diffusion coefficient to the particle size using the viscosity of the dispersion medium and some device constants is the Z-average particle size, which is a value suitable for the purpose of quality management as an indicator for the dispersion stability.

As the sample subjected to the measurement by dynamic light scattering, to obtain a highly reliable measurement value, for example, a dispersion obtained by dispersing 4.8 mg/mL of micelles in pure water is used. The dispersion used may be diluted 10-fold. The dispersion which has been purified once or more with a 0.22-μm syringe filter, preferably 0.45-μm and 0.22-μm syringe filters, is preferably subjected to the measurement.

The mechanism of action of HFm of this aspect is speculated as follows.

HFm is believed to act based on the principle which has been known as the EPR (Enhanced Permeability and Retention) effect of a solid tumor model. Moreover, HFm is a polymer micelle formed with a polymer, and it is believed that the halofuginone compound included inside is not easily degraded in the blood circulation or absorbed in cells or organs which are not the target. As a result, the HFm of this aspect is easily diffused/absorbed in the tumor cells, gradually releases the halofuginone compound in the tumor cells and tumor-specifically suppresses Nrf2, and thus the HFm can achieve tumor specificity and induce a side-effect-free therapeutic reaction. Here, the invention is not restricted by the speculated mechanism above.

The HFm of this aspect is obtained, for example, by association of a plurality of the conjugates of the first aspect in an aqueous solution. At this point, the conjugates can aggregate in the aqueous solution using the hydrophobic interaction of the halofuginone compound as one driving force. As a result, it is believed that the relatively hydrophobic halofuginone compound is housed in the micelle, the relatively hydrophilic PEG chain block forms the micelle shell, and the polypeptide chain block connects the shell and the inside to form the target micelle. In this regard, however, the HFm of this aspect is not limited only to the micelle formed by the mechanism but may be a micelle formed by another mechanism.

### <<Anticancer Agent>>

A third aspect of the invention is an anticancer agent containing the conjugate of the first aspect or the micelle (HFm) of the second aspect as an active ingredient. The anticancer agent may be a pharmaceutical composition containing the conjugate of the first aspect or the micelle of the second aspect as an active ingredient.

### (Cancer)

The type of the cancer to which the anticancer agent of this aspect is applied is not particularly limited, and examples thereof include malignant melanoma, malignant lymphoma, gastrointestinal cancer, lung cancer, lung adenocarcinoma, thymic carcinoma, esophageal cancer, stomach cancer, large bowel cancer, rectal cancer, colon cancer, ureteral tumor, gallbladder cancer, cholangiocarcinoma, biliary tract cancer, breast cancer, liver cancer, pancreatic cancer, spleen cancer, testicular tumor, maxillary cancer, tongue cancer, lip cancer, oral cancer, pharyngeal cancer, laryngeal cancer, ovarian cancer, uterine cancer, prostate cancer, thyroid cancer, brain tumor, Kaposi's sarcoma, hemangioma, leukemia, polycythemia vera, neuroblastoma, retinoblastoma, myeloma, bladder tumor, sarcoma, osteosarcoma, myosarcoma, skin cancer, basal cell carcinoma, cutaneous appendage carcinoma, and skin metastasis. The anticancer agent can be applied not only to malignant tumors but also to benign tumors.

The cancer to which the anticancer agent of this aspect is applied is not particularly limited, and the anticancer agent can be applied to a cancer in which activation of Nrf2 is observed.

The cancer to which the anticancer agent of this aspect is applied is not particularly limited, and the anticancer agent can be applied to a cancer in which mutation of Keap1 or Nrf2 is observed.

### (Administration of Anticancer Agent)

When the anticancer agent of this aspect is used, the dose of the anticancer agent is appropriately determined according to the conditions, such as the age, the gender, the body weight, and the symptom of the subject of administration, and the route of administration, and is not particularly limited. In general, the daily dose of the active ingredient for an adult is in the range of around 1 μg/kg to 10 mg/kg. The anticancer agent at the dose may be administered once a day or may be administered or continuously administered in several divided portions (for example, around twice or three times).

The subject of administration of the anticancer agent of this aspect is a human or an animal. The administration form of the anticancer agent of this aspect may be oral administration or parenteral administration such as intravenous, intramuscular, subcutaneous, or intradermal injection, rectal administration, and transmucosal administration. Parenteral administration is preferable, and intravenous administration is more preferable. Examples of the preparation form suitable for oral administration include tablets, pills, granules, powder, capsules, a liquid agent, suspension, emulsion, syrup, and the like, and examples of the pharmaceutical composition suitable for parenteral administration include injection, infusion, nasal drops, spray, inhalant, suppositories, percutaneous absorbents such as ointment, cream, powdery liniment, liquid liniment, and patches, and the like. The pharmaceutical composition suitable for parenteral administration containing this aspect is preferably injection. The non-human animal is preferably a mammal.

### (Other Composition Contained in Anticancer Agent)

When the anticancer agent of this aspect is a solid preparation such as tablets, pills, powder, powdery preparation, and granules, such a solid preparation is produced by appropriately mixing the active ingredient with an appropriate additive such as an excipient such as lactose, sucrose, D-mannitol, cornstarch, synthetic or natural gum, and crystalline cellulose, a binder such as starch, hydroxypropyl cellulose, hydroxypropyl methylcellulose, gum Arabic, gelatin, and polyvinylpyrrolidone, a disintegrant such as calcium carboxymethyl cellulose, sodium carboxymethyl cellulose, starch, cornstarch, and sodium alginate, a lubricant such as talc, magnesium stearate, and sodium stearate, and a filler or a diluent such as calcium carbonate, sodium carbonate, calcium phosphate, and sodium phosphate. The tablets and the like may be coated with sugar coating, gelatin, enteric coating, film coating, or the like using a coating agent such as hydroxypropyl methyl cellulose, sucrose, polyethylene glycol, and titanium oxide according to the need.

When the anticancer agent of this aspect is a liquid preparation such as injection, eye drops, nasal drops, inhalant, spray, lotion, syrup, a liquid agent, suspension, and emulsion, such a liquid preparation is adjusted as a sterilized aqueous solution, nonaqueous solution, suspension, liposome, emulsion, or the like by dissolving the active ingredient in purified water, an appropriate buffer such as phosphate buffer, saline, a physiological salt solution such as Ringer's solution and Locke's solution, a plant oil such as cacao butter, sesame oil, and olive oil, an organic solvent such as mineral oil, higher alcohols, higher fatty acids, and ethanol, or the like and, according to the need, appropriately adding an additive, such as an emulsifier such as cholesterol, a suspending agent such as gum Arabic, a dispersing agent, a wetting agent, a polyoxyethylene hydrogenated castor oil-based or polyethylene glycol-based surfactant, a solubilizing agent such as sodium phosphate, a stabilizer such as sugar, a sugar alcohol, and albumin, a preservative such as paraben, an isotonic agent such as sodium chloride, glucose, and glycerin, a buffer, a soothing agent, an adsorption inhibitor, a moisturizing agent, an antioxidant, a coloring agent, a sweetener, a flavor, and an aroma substance. At this point, the injection preferably has physiological pH.

When the anticancer agent of this aspect is a semi-solid preparation such as lotion, cream, and ointment, such a semi-solid preparation is produced by appropriately mixing the active ingredient with fat, fatty oil, lanolin, Vaseline, paraffin, wax, plaster, resin, plastic, a glycol, a higher alcohol, glycerin, water, an emulsifier, a suspending agent, or the like.

The active ingredient content of the anticancer agent of this aspect is not particularly limited. In general, the content based on the total mass of the anticancer agent of this aspect is 0.001 to 100 mass %.

### (Therapeutic Method of Cancer)

When the anticancer agent of this aspect is used, the anticancer agent of this aspect can also be used in combination with existing chemotherapy, surgical treatment, radiotherapy, thermotherapy, immunotherapy, or the like.

Another aspect of the invention is the production of an anticancer agent. The conjugate of the first aspect and the micelle of the second aspect are useful as a material for producing the anticancer agent of the third aspect.

Another aspect of the invention is use in cancer therapy. The conjugate of the first aspect and the micelle of the second aspect are useful for use in cancer therapy.

Another aspect of the invention is a method for treating a cancer. An example thereof is a method for treating a cancer including administering a therapeutically effective amount of the conjugate of the first aspect and the micelle of the second aspect to a patient in need of the treatment.

### EXAMPLES

Although details of the invention are explained with Test Examples below, the invention is not limited to the Test Examples below.

### <Test Method>

### (Reagents)

Halofuginone hydrobromide (HF, CAS No. 64924-67-0) was purchased from Carbosynth Limited (code no. FH23731, Berkshire, UK). An undiluted HF solution was prepared at a concentration of 10 mg/mL using dimethyl sulfoxide (DMSO). The undiluted solution was further diluted with saline. α-Methoxy-ω-aminopropyl poly(ethylene glycol) having a molecular weight of 12 kDa was purchased from NOF CORPORATION (Tokyo, Japan). β-Benzyl-L-aspartic acid N-carboxy-anhydride (BLA-NCA) was purchased from CHUO KASEIHIN CO., INC (Tokyo, Japan).

### (Synthesis of HF-Modified PEG-PAsp (PEG-PAsp(HF)))

Poly(ethylene glycol)-poly((3-benzyl-L-aspartate) (PEG-PBLA) was synthesized in the same manner as in Biomacromolecules, 2020, 21(10):4365-4376 using α-methoxy-ω-aminopropyl poly(ethylene glycol) (NOF CORPORATION, Tokyo) as a macro-initiator by ring cleavage polymerization of BLA-NCA.

In this test example, 500 mg of α-methoxy-ω-aminopropyl poly(ethylene glycol) and 456 mg of BLA-NCA (CHUO KASEIHIN CO., INC, Tokyo) were used for the PEG-PBLA polymerization. The polymerization degree of the PBLA segment in PEG-PBLA was determined to be 40 from a ¹H nuclear magnetic resonance (NMR) spectrum (solvent: DMSO-d₆, temperature: 80° C.) (JNM-ECS 400, JEOL Ltd., Tokyo).

PEG-PBLA was dissolved in a 0.5 M aqueous NaOH solution (five equivalents based on BLA), and deprotection was conducted at room temperature over one hour.

Next, the reaction mixture was poured to a dialysis membrane (MWCO: 12-14 kDa), dialyzed with a 0.01 M aqueous HCl solution for half a day and with deionized water for half a day and then freeze-dried. The obtained PEG-PAsp was dissolved in N,N-dimethylformamide (10 mg/mL). In the PEG-PAsp solution, 2-methyl-6-nitrobenzoic anhydride (1.2 equivalents based on Asp) and 2,2-dimethoxypropane (1.5 equivalents based on Asp) were mixed and stirred at 25° C. for 20 minutes. HF was dissolved in DMF separately (1.0 equivalent based on Asp, 6 mg/mL), and the solution was added to the PEG-PAsp solution. The mixture was protected from light and stirred for one day.

The mixture was poured to a dialysis membrane (MWCO: 6-8 kDa), dialyzed with deionized water for two days and protected from light to form micelles. The dialysate was caused to pass through a 0.45-μm syringe filter, concentrated by ultrafiltration (MWCO: 30000) and further filtered with a 0.22-μm membrane for sterilization.

By dynamic light scattering using Zetasizer Nano (Malvern Instruments, Worcestershire, UK) having a He—Ne laser, the diameter and the size distribution (polydispersity index) of the obtained PEG-PAsp(HF) micelles were determined to be 38 nm and 0.12, respectively. One milliliter of the micelle solution was sampled and freeze-dried, and the micelle concentration was adjusted. The HF-carrying amount was calculated. The weight of the dried powder was measured, and the powder was dissolved in DMSO-d₆ (2% D2O and 0.05% TMS). The solution was subjected to ¹H NMR measurement (temperature: 80° C.). The HF-carrying amount was determined to be 14.4 per polymer from the peak intensity ratio of the PEG proton (1090 H) to the benzyl proton (43.2 H) of HF. The PEG-PAsp(HF) micelle solution was stored at −80° C. until use.

### (Cell Culture)

WT-GFP and Keap1-mCherry Hepa1 cells were prepared in the same manner as in Mol Cell Biol., 2020 Oct 26, 40(22). The cells were maintained in Dulbecco's modified Eagle's medium (DMEM) containing 10% (v/v) fetal bovine serum and antibiotics (penicillin-streptomycin) at 37° C. and 5% CO₂.

### (Cell Fluorescence Analysis)

For cell fluorescence analysis, 4×10⁴ WT-GFP cells and 2×10⁴ Keap1-mCherry Hepa1 cells were cultured separately on day −1 in a black 96-well plate (Corning; number 3904). On day 0, HF or saline was introduced at various concentrations, and then the plate was returned to an incubator at 37° C. until day 2. The fluorescence intensities of the cells were measured using a PHERAstar FS microplate reader (BMG Labtech, Ortenberg, Germany).

### (Immunoblotting)

For analyzing cultured cells, 1×10⁶ WT-GFP and Keap1-mCherry Hepa1 cells were separately seeded on day −1 and treated with 50 nM HF on the next day (day 0). Whole cell lysates were prepared by lysing the cells with SDS buffer (0.25 M Tris-HCl (pH 6.8), 8% (w/v) SDS, and 20% glycerol) and collected 0, three, six, 12, and 18 hours after the HF treatment. An anti-Nrf2 antibody (Cell Signaling Technology, #12721, 1:500 dilution) and an anti-α-tubulin antibody (Sigma, T9026, 1:1000 dilution) were used.

### (Mouse)

Keap1^{FB/FB}:Kras^{G12D} mice were produced in the same manner as in Cancer Res, 15 Aug. 2020, 80(16):3331-3344, Genes & Dev., 2001, 15:3243-3248, and Am J Respir Cell Mol Biol., 2010 May, 42(5):524-36. All the mice used in this test had the C57BL/6J background. Saline, HF, and HFm were intravenously injected to the mice. All the in vivo experiments were approved by the animal welfare committee of Tohoku University.

### (Intranasal Administration of Cre Recombinase-Expressing Adenovirus (Adeno-Cre))

Adeno-Cre was purchased from VectorBioLabs (Ad-Cre-GFP, catalog no. 1700). Adeno-Cre was administered in the same manner as in Nat Protoc 4, 2009, 1064-1072. Briefly, 1×10⁷ plaque-forming units of Adeno-Cre were mixed with Eagle's minimum essential medium (MEM) and calcium chloride. The mixture was incubated on ice for 20 minutes and used for intranasal dropping within one hour of the preparation. Droplets containing the Adeno-Cre mixture were administered to the nostrils of anesthetized mice (eight to 12 weeks old) using a pipette. The mice were allowed to inhale the droplets gradually.

### (Flow Cytometry)

Monocytes were isolated from the bone marrow, the spleen, and the thymus using Ficoll-Paque PREMIUM 1.084 (Cytiva 17-5446-02). The monocytes were stained with allophycocyanin-eFluor 780 conjugate anti-c-Kit (Invitrogen; Clone 2B8), Brilliant Violet 421 conjugate anti-B220 (Biolegend; Clone RA2-6B2), phycoerythrin conjugate anti-F4/80 (Biolegend; Clone BM8), fluorescein isothiocyanate conjugate anti-Ly6G (Biolegend; Clone 1A8), Brilliant Violet 421 conjugate anti-CD11b (Biolegend; Clone M1/70), fluorescein isothiocyanate conjugate anti-CD71 (eBiosciences; CloneR17217), phycoerythrin conjugate anti-Ter119 (eBiosciences; Clone TER-119), PE-Cyanine7 conjugate anti-CD4 (Invitrogen; CloneGK1.5), and allophycocyanin conjugate anti-CD8 (Biolegend; Clone S3-6.7). The dead cells were removed using propidium iodide staining. The analysis was made using FACSVerse (BD Biosciences). The data were analyzed using FlowJo software (BD Biosciences).

### (Liquid Chromatography-Tandem Mass Spectrometer (LC-MS/MS) Analysis)

HF was measured as in Drug Test. Anal., 2016, 8, 465-476 with slight modification. Briefly, about 30 mg of a frozen tissue was put into a 2-mL plastic tube, and 600 μL of acetonitrile/2-isopropanol (3:2, v/v), 15 mg of anhydrous sodium acetate, and 10 mg of anhydrous magnesium sulfate were added. As the internal standard, HF-13C6 HBr (Honeywell/Fluka, Germany) was used. The sample was mixed vigorously for 50 seconds, homogenized in an ultrasonic bath for 10 minutes and then shaken for 30 minutes. The mixture was centrifuged at 16000×g for 10 minutes. The supernatant was diluted with the equivalent amount of water and centrifuged at 16000×g for 10 minutes. The supernatant in a volume of 30 μL was introduced to an ultra-high performance liquid chromatography (UHPLC)-MS/MS system.

The UHPLC-MS/MS analysis was conducted with Acquity Ultra Performance LC I-class system (Waters) having a binary solvent manager, a sample manager, and a column heater connected to Waters Xevo TQ-S MS/MS system having an electrospray ionization device operated by cation mode. The MS/MS was conducted by the Multiple reaction monitoring mode. The capillary voltage was 3.5 kV, and the cone voltage was 120 V. The cone gas flow rate was 150 L/h, and the source offset and the temperature were set at 50 V and 150° C., respectively. The desolvation temperature was set at 500° C., and the desolvation gas flow, the impinging gas flow, and the spray gas flow were 1000 L/h, 0.15 ml/min, and 7.00×10⁵ Pa, respectively. Nitrogen was used for both the cone gas and the spray gas. Using a reverse phase column (Acquity UPLC BEH C18; 100 mm×2.1 mm [inside size], 1.7 μm particle size; Waters Corp.), LC separation was conducted with gradient elution with solvent A (0.1% formic acid/water, v/v) and solvent B (100% acetonitrile) at 0.5 ml/min. The initial condition was set at 5% solvent B, which was maintained for one minute. The solvent B was increased linearly to 95% over four minutes, and the condition was maintained for two minutes. At the end, the mobile phase was returned to the initial condition, and this state was maintained for two minutes until the completion of the run. The column oven temperature was 50° C. MassLynx v4.1 software (Waters) was used for data collection, and Traverse MS v1.2.7 software (Reifycs) was used for data analysis.

### (Histology)

The lung tissues were fixed in 10% formalin (Mildform 10N; FUJIFILM Wako Pure Chemical Corporation, Osaka) and embedded in paraffin. The slices were stained with hematoxylin-eosin (HE) or elastica-Masson (EM). To measure the tumor areas, five slices were cut out with a certain distance and stained with HE. The tumor areas were measured using ImageJ software (NIH).

### Test Example 1: HF Suppresses Nrf2 Accumulation In Vitro and Thus Targets Nrf2-Activated Cancer Cells

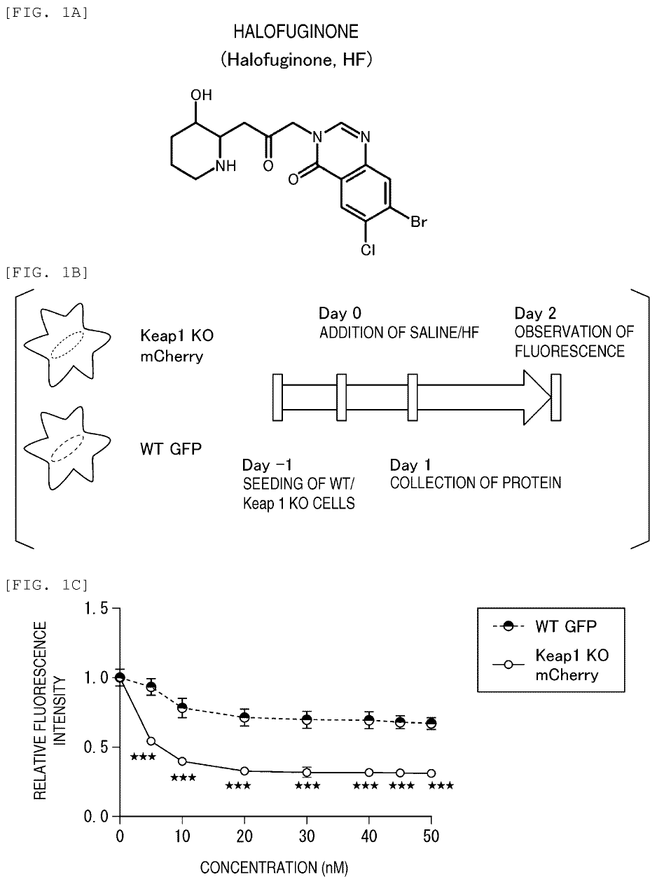

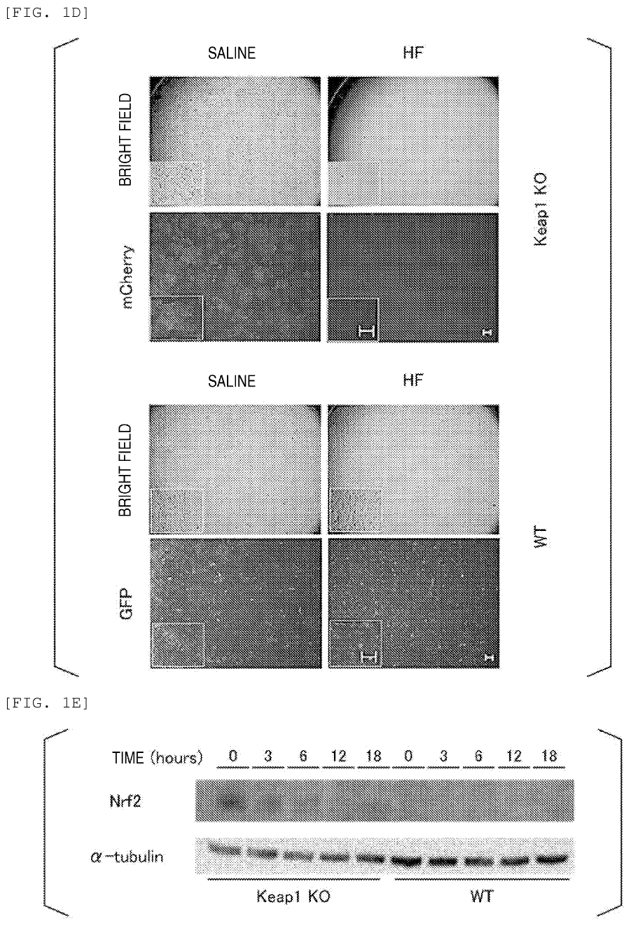

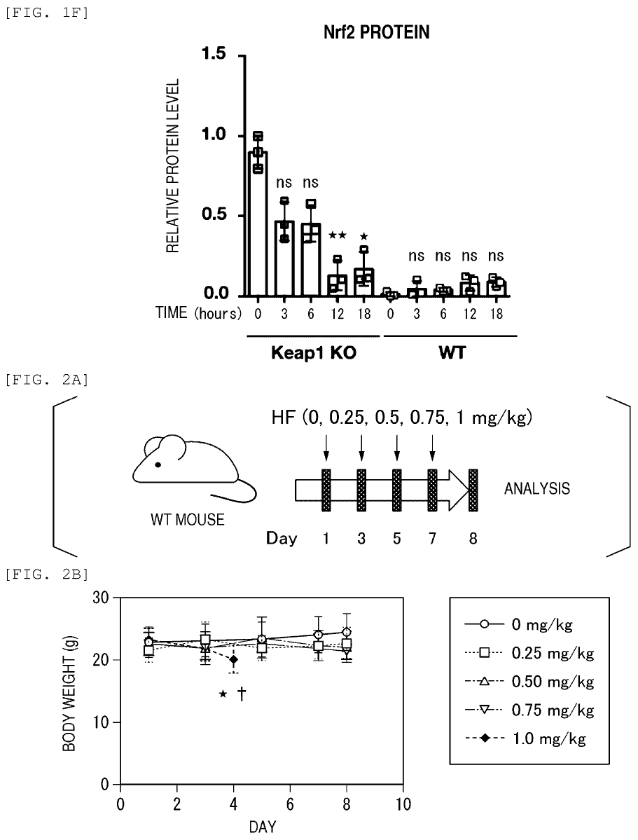

HF has been reported to suppress Nrf2 accumulation in cells (FIG. 1A). To examine the tumor suppressive role of HF in Nrf2-activated tumor cells, enhanced green fluorescent protein (EGFP)-expressing wild type (WT) Hepa1 cells (hereinafter also referred to as “WT-GFP cells”) and mCherry-expressing Keap1 knockout cells (hereinafter also referred to as “Keap1 KO-mCherry cells”) were treated with HF. Both isogenic cell lines used were the same as those in Mol Cell Biol., 2020 Oct 26, 40(22). Keap1 is a negative regulator of Nrf2, and when Keap1 is knocked out, Nrf2 is activated constitutively. As shown in FIG. 1B, the fluorescence intensities (the values relative to those of the saline-treated cells) were measured 48 hours after the cells were exposed to HF. It has been shown in Mol Cell Biol., 2020 Oct 26, 40(22) that there is a correlation between the relative fluorescence intensity and the cell survival rate. When the Keap1 KO-mCherry cells were treated with HF at various doses up to 50 nM, the count of the Keap1 KO-mCherry cells decreased significantly in a concentration-dependent manner, but the count of the WT-GFP cells did not decrease substantially with the increase in the HF concentration (FIG. 1C). FIG. 1D shows typical images of the Keap1 KO-mCherry cells and the WT-GFP cells which were treated with 50 nM HF. From the images, it was shown with good reproducibility that while the count of the Keap1 KO-mCherry cells decreases with HF treatment, the count of the WT-GFP cells does not substantially decrease. The above results clearly show that HF influences the survival rate of the Nrf2-activated Keap1 KO-mCherry cells more strongly than the survival rate of the WT-GFP cells.

To examine whether the selective decrease in the Keap1 KO-mCherry cells was caused by suppression of Nrf2 accumulation, Nrf2 protein was analyzed by immunoblotting at various points in time after isogenic Hepa1 cells were exposed to HF. In particular, in the Keap1 KO-mCherry cells, Nrf2 protein depleted rapidly with HF treatment (FIG. 1E). On the other hand, Nrf2 protein was not detected at all the points in time in the WT-GFP cells. The above experiment was repeated, and densitometry measurement of immunoblotting bands was conducted (FIG. 1F). The above results demonstrate that HF significantly suppresses the viability of Nrf2-activated cancer cells through rapid depletion of Nrf2 protein.

### Test Example 2: Evaluation of Dose-Dependent In Vivo Toxicity of HF

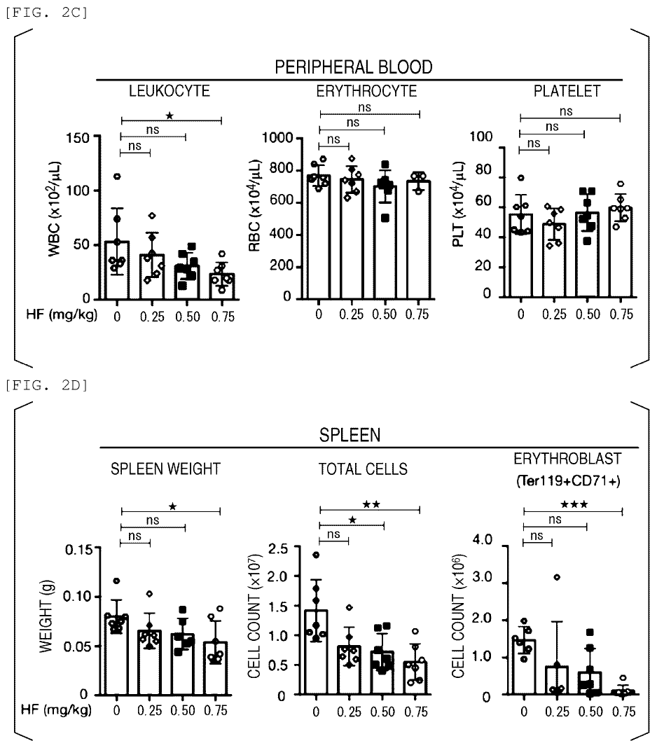

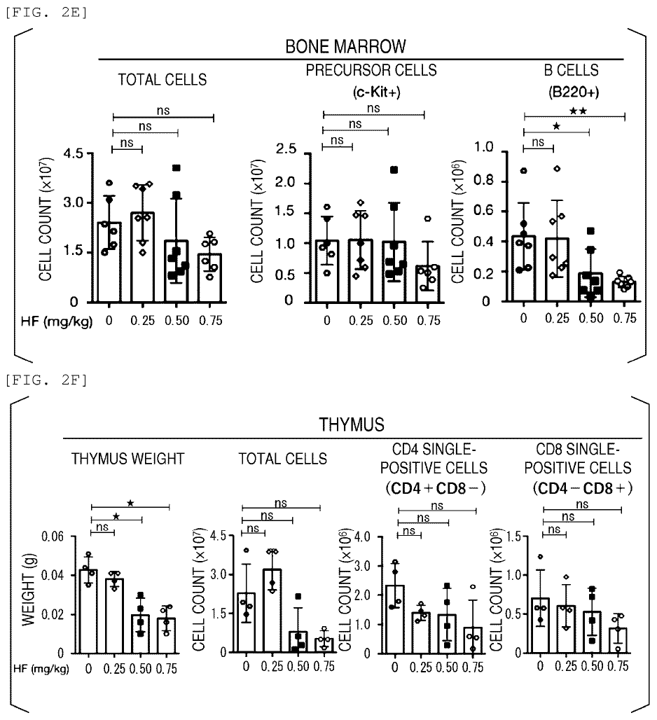

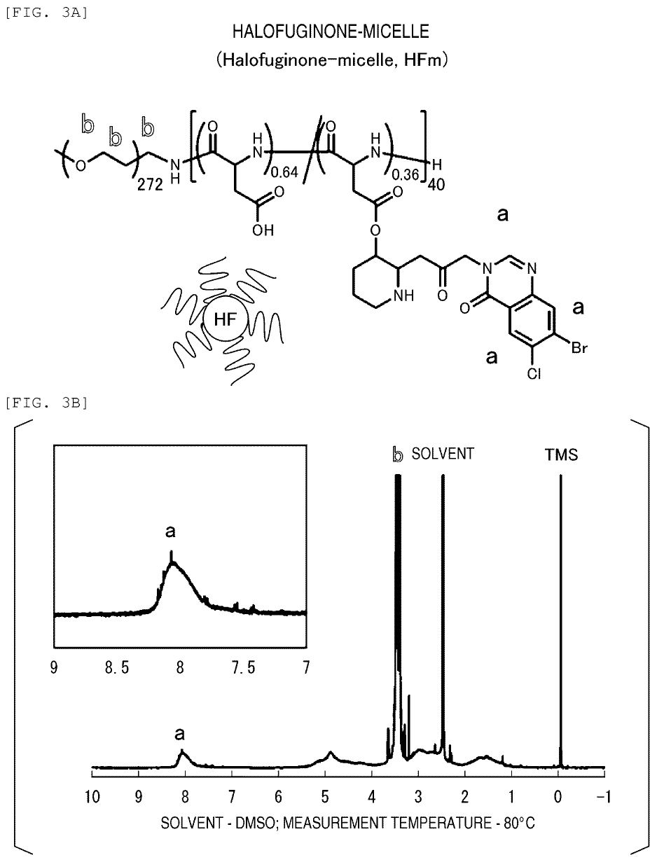

Preclinical animal testing was conducted to determine the maximum tolerated dose (hereinafter also referred to as MTD) of HF. Preceding experiment using a patient-derived xenograft mouse model was conducted at a HF dose of 0.25 mg/kg body weight (kg bw), and HF at this dose did not show any sign of acute side effects in mice (Free Radical Biology and Medicine, 2017, 103, 236-247). Thus, in this test, HF was administered to WT C57BL/6 mice (eight to 12 weeks old, both females and males) incrementally in the range of 0.25 mg/kg to 1 mg/kg (FIG. 2A). HF was administered to the tail veins every two days four times in total.

As a result, eight mice in total to which 1 mg/kg of HF was administered showed a sign of acute disorder after two injections and were moribund. Moreover, the body weights of the mice decreased significantly (−13.4%). Because one mouse in the administration group died after the second administration, the remaining mice in the administration group were euthanized before the completion of the experimental protocol on the fourth day (FIG. 2B) These mice were excluded from the subsequent analysis.

After HF was intravenously injected four times, the blood cell markers in the peripheral blood were examined. As a result, the leukocyte count decreased in a dose-dependent manner after the HF administration, but the erythrocyte counts and the platelet counts were equivalent in all the groups (FIG. 2C). To examine whether HF would have an immunosuppressive effect on different cell groups, spleen, thymus, and bone marrow samples were also analyzed by flow cytometry. As a result, it was found that HF significantly exhausts different hematopoietic cells and immune cells in a dose-dependent manner. Through the HF injection, the whole cells and the erythroblasts (Ter119⁺CD71⁺) of the spleen decreased largely in a dose-dependent manner (FIG. 2D). The decrease in the erythroblasts was extremely remarkable, and most of the erythroblasts disappeared after the administration of 0.75 mg/kg of HF. The spleen weight also decreased stepwisely with the increase in the HF dose.

In the bone marrow, the total cell count and the precursor cell count (c-Kit⁺) did not change largely, but the B cell count (B220⁺) was decreased by the administration of 0.75 mg/kg of HF (FIG. 2E). When HF was administered, the thymus weight decreased with significance in a dose-dependent manner (FIG. 2F). In the thymus, the total cells of the thymus and the CD4 single-positive (CD4⁺CD8⁻) cells decreased, and the CD8 single-positive (CD4⁻CD8⁺) cells also had a tendency towards a decrease.

The immune cell and hematopoietic cell groups decreased simultaneously with significant reduction in the tissue sizes of spleen and thymus.

### Test Example 3: Analysis of Pharmacokinetics and In Vivo Distribution of HF-Carrying Polymer Micelles (HFm)

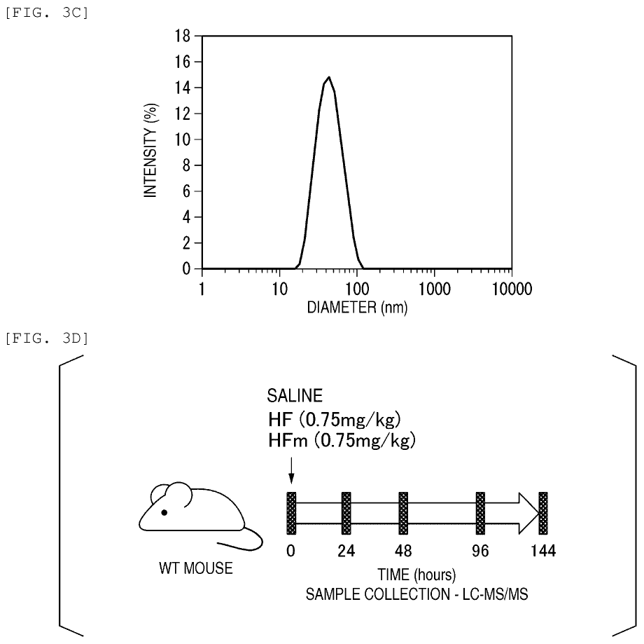

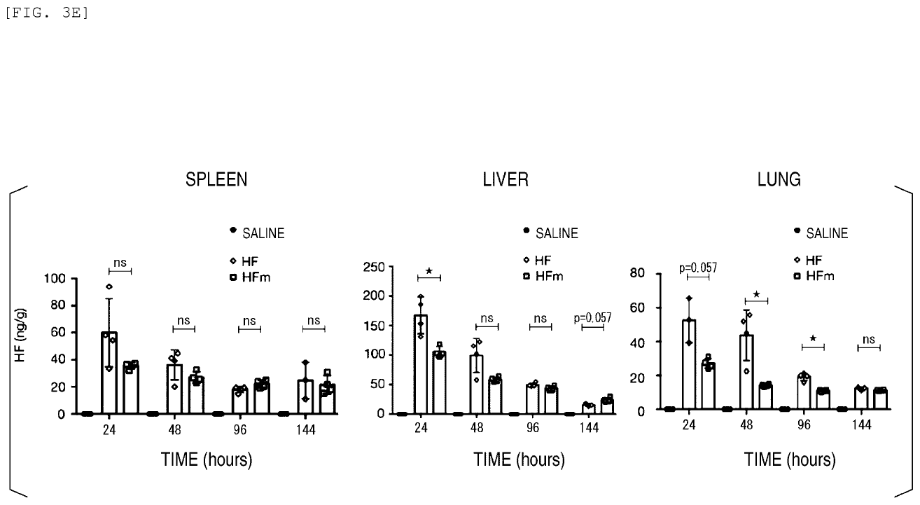

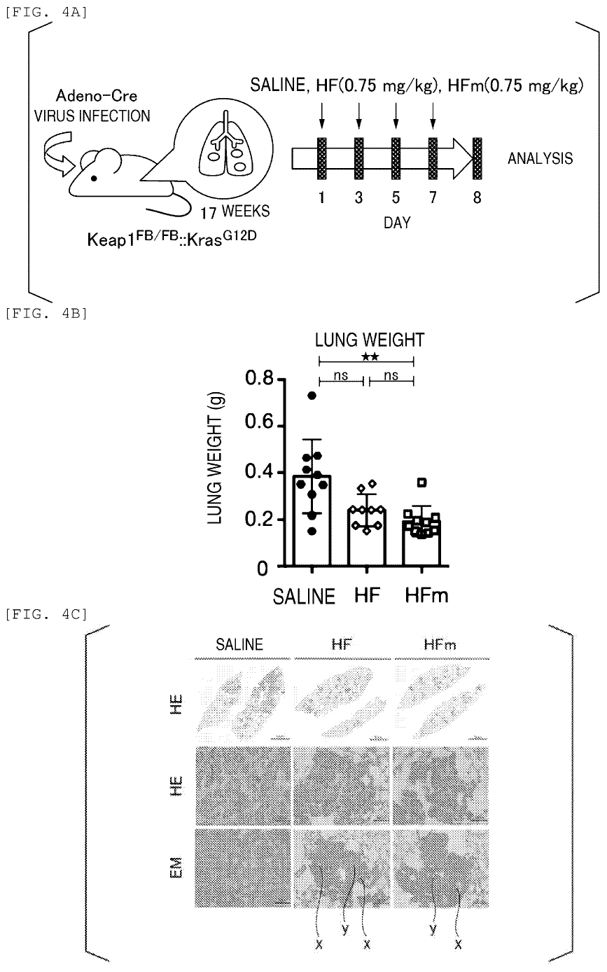

From the above results, it was suggested that HF causes serious systemic toxicity in the WT in vivo model. Thus, it was supposed that delivering HF to a tumor as nanomedicine would be a persuasive approach to cut off the side effects from the therapeutic reaction and remove the restriction. Accordingly, in this test, a new type of polymer micelle (HFm) in which HF is incorporated with an ester bond of HF and PEG-PAsp was developed (FIG. 3A). A peak corresponding to HF was detected in a ¹H NMR spectrum, and it was found that 14.4 molecules of HF were included per one molecule of the polymer (FIG. 3B, peak a). Moreover, the peak b in the spectrum corresponds to the protons of the polymer (FIG. 3B). By dynamic light scattering, the hydrodynamic diameter of HFm was determined to be 38 nm, and the size distribution (polydispersity index) was determined to be 0.12 (FIG. 3C).

To elucidate the pharmacokinetic characteristics of HFm, LCMS/MS test was conducted using WT C57BL/6 mice. saline, HF (0.75 mg/kg bw), or HFm (0.75 mg/kg bw) was administered once to eight- to 12-week-old WT mice. The HFm amount was the net weight of HF excluding the polymer weight. The above mice were followed until 144 hours after the administration, and spleen, liver, and lung samples were collected at different points in time (FIG. 3D). Through determination of the HF amounts by LCMS/MS analysis, it was found that the accumulated HF amounts in the three tissues were lower after the HFm administration than after the HF administration (FIG. 3E). However, the accumulated HF amounts of the HF administration group and the HFm administration group were equivalent at the end of the test (144 hours). This suggests that encapsulation of HF in polymer micelles leads to gradual release of HF from HFm. However, it is highly likely that HF was not released and remained in the core of HFm even after 144 hours. Due to the restrictions on the test, HF which was still remaining in HFm could not be identified. A large amount of HF is accumulated immediately when free HF is administered, and it is thus speculated that HF administration causes high systemic toxicity.

### Test Example 4: HF/HFm Administration Suppresses Progress of Nrf2-Activated Tumor

To examine whether administration of HF or HFm could suppress the progress of Nrf2-activated non-small-cell lung cancer (NSCLC), a Kras-driven lung adenocarcinoma mouse model was used (Genes&Dev., 2001, 15:3243-3248). A mouse having a loxP-Stop-loxP Kras^{G12D}-knocked-in allele (hereinafter also referred to as “Kras mouse”) was mated with a mouse having a Keap1-flox allele in the loxP site including the third and fourth exons (hereinafter also referred to as “Keap1^{FB}”) (Am J Respir Cell Mol Biol., 2010 May, 42(5):524-36), and thus a Keap1^{FB/FB}:Kras^{G12D} complex mouse was obtained (Cancer Res, 15 Aug. 2020, 80(16):3331-3344). Through Cre recombination by intranasal infection with Adeno-Cre, activation of Kras cancer gene and deletion of Keap1 exons were achieved simultaneously. By this operation, Nrf2-activated lung adenocarcinoma was successfully imitated in mice.

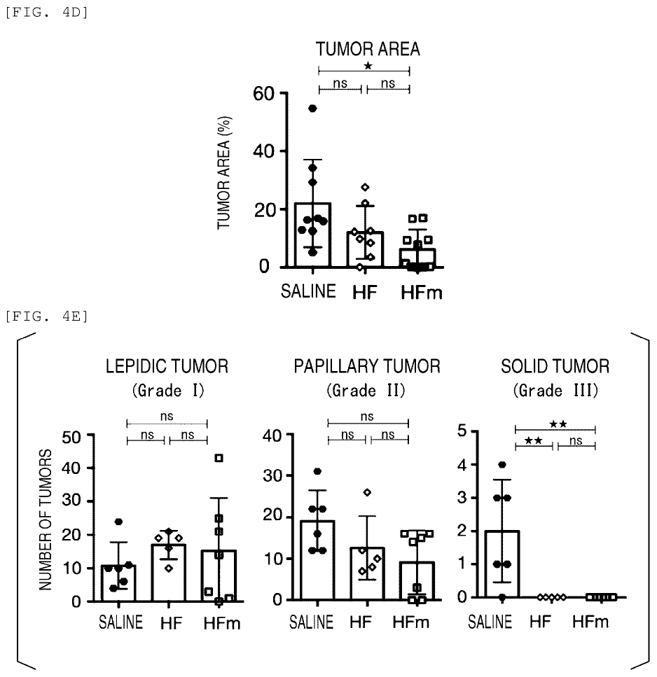

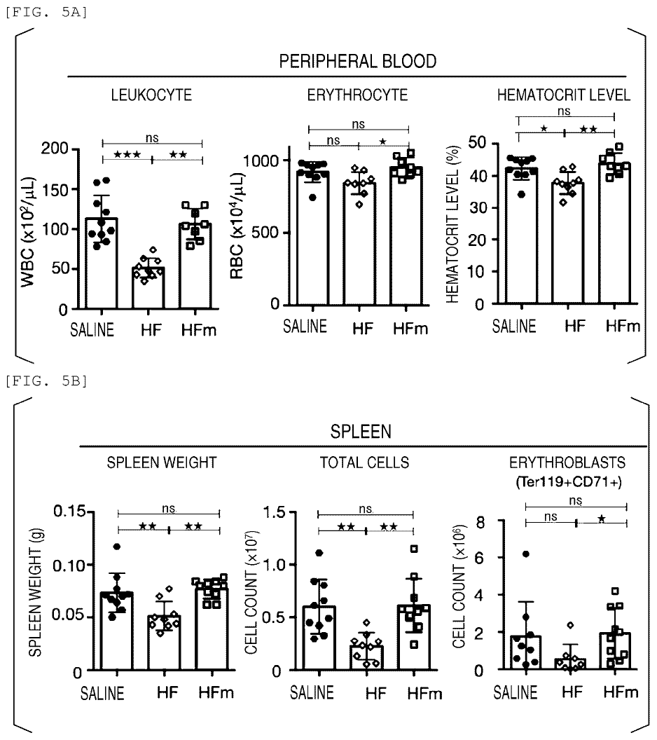

Then, after the tumor was grown for 17 weeks, administration of HF or HFm was started. HF or HFm was intravenously administered every two days, and injection was conducted four times in total. Because the MTD of HF was determined to be 0.75 mg/kg bw (FIG. 2), it was decided to use 0.75 mg/kg bw of HF or HFm in this cancer-bearing mouse experiment. The mice were sacrificed on the eighth day. Various characteristics of the lung tumors were analyzed, and the effectiveness of HF and HFm changing the tumor burden was evaluated (FIG. 4A).

First, the lung weights were measured. The lung weights of the HF- and HFm-treated mice were lower with significance than the lung weights of the saline-treated mice (FIG. 4B). Next, lung slices were prepared and stained with HE (FIG. 4C, top). The dark stained parts in the HE-stained slices correspond to the tumors, and the tumor areas of the lungs of the mice treated with HF or HFm decreased significantly compared to the tumor areas of the lungs of the mice treated with saline. Similarly, the sizes of the respective tumors also decreased with the HF or HFm treatment (FIG. 4C, middle). When the dark stained areas were measured using image processing software, the tumor areas were decreased to almost a half (21% of the saline-treated group→10% of the HF-treated group) or to one third (21% of the saline-treated group→6% of the HFm-treated group) by the HF or HFm treatment (FIG. 4D).

Next, by EM staining of the lung slices, the influence of HF and HFm on the malignancy of tumors was examined (FIG. 4C, bottom). Using images of the slices, the lung lesions were evaluated according to the guidelines of the system of the 2015 World Health Organization Classification of Lung Tumors (Front Oncol., 2017 Aug 28, 7: 193). Briefly, lung tumors include two types, namely a lepidic lesion and a papillary lesion. A lepidic lesion indicates in situ proliferation of cells along the alveolar walls (arrows with “x”), while a papillary lesion indicates proliferation away from the alveolar walls (arrows with “y”). A tumor with many papillary lesions is called Grade II, and it is known that the prognoses of a patient having many papillary tumors is poor compared to that of a patient having many lepidic tumors (Grade I). When the main component of the tumor is composed of a solid sheet and when no recognizable lepidic or papillary pattern is found, the tumor is classified as a solid tumor (Grade III).

The numbers of the lepidic tumors and the papillary tumors in the lung slices of the Keap1^{FB/FB}:Kras^{G12D} complex mice which were treated with saline, HF, or HFm were counted. As a result, equivalent numbers of lepidic (Grade I) and papillary (Grade II) tumors were found in all the three groups (FIG. 4E). It was shown that the frequency of the papillary tumors was lower in the tumor model mice treated with HF or HFm than in the control treated with saline. On the other hand, the solid adenocarcinomas of Grade III disappeared completely after the HF or HFm treatment. The above results clearly show that both HF and HFm can prevent progress of Nrf2-activated lung cancer to a higher-grade solid cancer.

### Test Example 5: HFm Relieves the Side Effect of Systemic Immunosuppression

It was found that HF treatment not only promotes a severe side effect but also causes considerable cell deaths and systemic immunosuppression to damage healthy tissues. Because HFm exhibited an equivalent or higher effect on lung adenocarcinoma than that of HF in the above test, the function of HFm to overcome the systemic side effect of HF in cancer-bearing mice was next evaluated. Because HF is slowly and continuously released from HFm through encapsulation of HF in polymer micelles, it was supposed that HFm may act as a far safer drug than HF. Thus, because the MTD of HF was 0.75 mg/kg bw, various side effect markers were examined in cancer-bearing model mice treated with 0.75 mg/kg bw of HFm and compared with those of the mice treated with the same amount of HF.

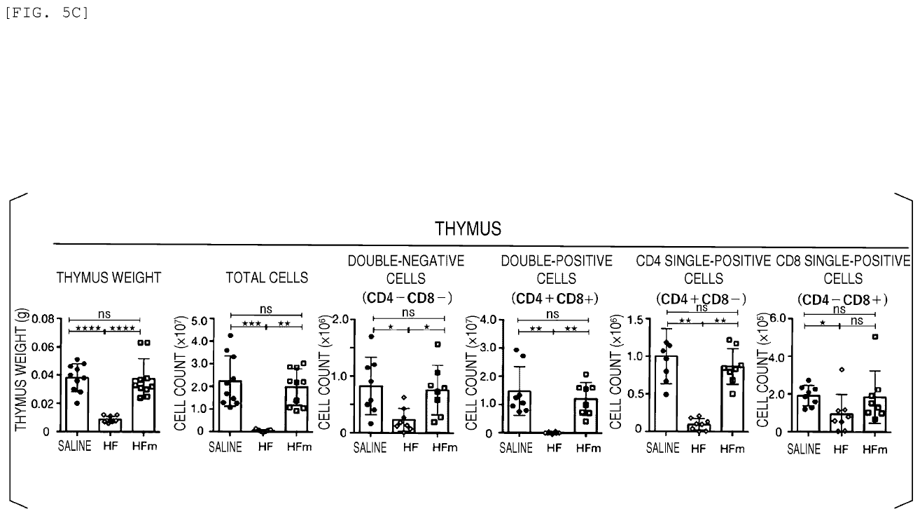

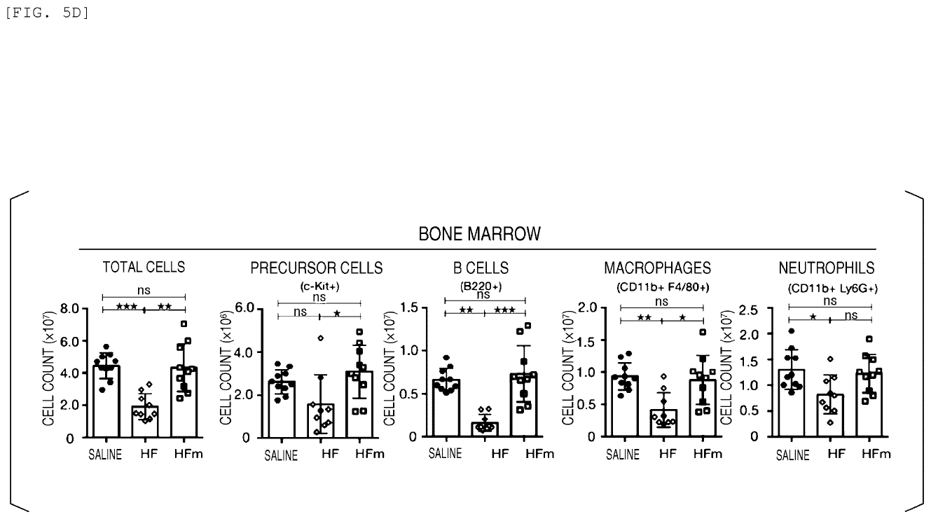

First, the indicators of peripheral blood were measured. Although a significant decrease in the leukocyte count was observed in the mice treated with HF, such a decrease was not observed in the mice treated with HFm, and the leukocyte count of the HFm-treated mice was equivalent to that of the saline-treated mice (FIG. 5A). Although the erythrocytes and the hematocrit levels of the mice treated with HF decreased, and a sign of anemia was observed, such a decrease was not observed in the HFm-administered mice (FIG. 5A). Next, when the spleen weights were examined, the spleen weight was decreased with significance by the HF treatment, but the decrease was not observed in the HFm-treated mice (FIG. 5B). The spleen cell count was decreased with significance by the HF treatment but was not decreased by the HFm treatment. This shows that the decrease in the spleen weight observed in the HF-treated mice was caused by a significant decrease in the absolute number of the spleen cells. Similarly, although HFm did not cause any clear decrease in the erythroblasts (Ter119⁺CD71⁺) of the spleen, the cell group was decreased with significance by the HF treatment. The above results show that HFm relieves the severe adverse effect of HF on the hematopoietic system with significance.

It was also found that atrophy of the thymus was remarkable in the mice treated with HF and that the total cell count decreased significantly (FIG. 5C). The atrophy of the thymus was not observed at all with the HFm treatment, and the total cell count of the thymi of the HFm-treated mice was equivalent to that of the saline-treated mice. To examine the reasons for the significant decreases in the thymus tissue weight and the cell count, the major cell types in the thymus were determined using flow cytometry. The double-negative cells (CD4⁻CD8⁻), the double-positive cells (CD4⁺CD8⁺), the CD4 single-positive cells (CD4⁺CD8⁻), and the CD8 single-positive cells (CD4⁻CD8⁺) were examined. In the cancer-bearing mice treated with HF, the counts of the subgroups of the double-negative cells (CD4⁻CD8⁻), the double-positive cells (CD4⁺CD8⁺), and the CD4 single-positive cells (CD4⁺CD8⁻) of the immune cells decreased. On the other hand, such decreases in the thymus were not observed with the HFm treatment, and the cell counts were equivalent to those of the mice treated with saline. The CD8 single-positive cells (CD4⁻CD8⁺) of the mice treated HF also showed a tendency towards a lower frequency than those of the mice treated with saline or HFm. Interestingly, the decrease in the cell count was greater in immature cells (namely, the CD4⁻CD8⁻ cells and the CD4⁺CD8⁺ cells) than in the differentiated CD8 and CD4 single-positive cells (FIG. 5C).

Although the total cell count of the bone marrow was also decreased significantly by the administration of free HF, the decrease was not observed with the administration of HFm (FIG. 5D). In analysis with flow cytometry, the counts of the precursor cells (c-Kit⁺), the B cells (B220⁺), the macrophages (CD11b⁺F4/80⁺), and the neutrophils (CD11b⁺Ly6G⁺) decreased significantly in the bone marrows of the mice treated with HF, compared to those of the bone marrows of the mice treated with HFm or saline (FIG. 5D). The above results showed that the tolerability of HFm is high and that HFm does not at all cause the systemic side effect observed after the HF administration, in particular, severe atrophy of the thymus, decreases in the spleen size and the spleen cell count, and a decrease in the peripheral leukocyte count, which are the characteristics of the systemic immunosuppression.

### Test Example 6: HFm Treatment Decreases the Accumulated HF Amounts in Nonneoplastic Tissues

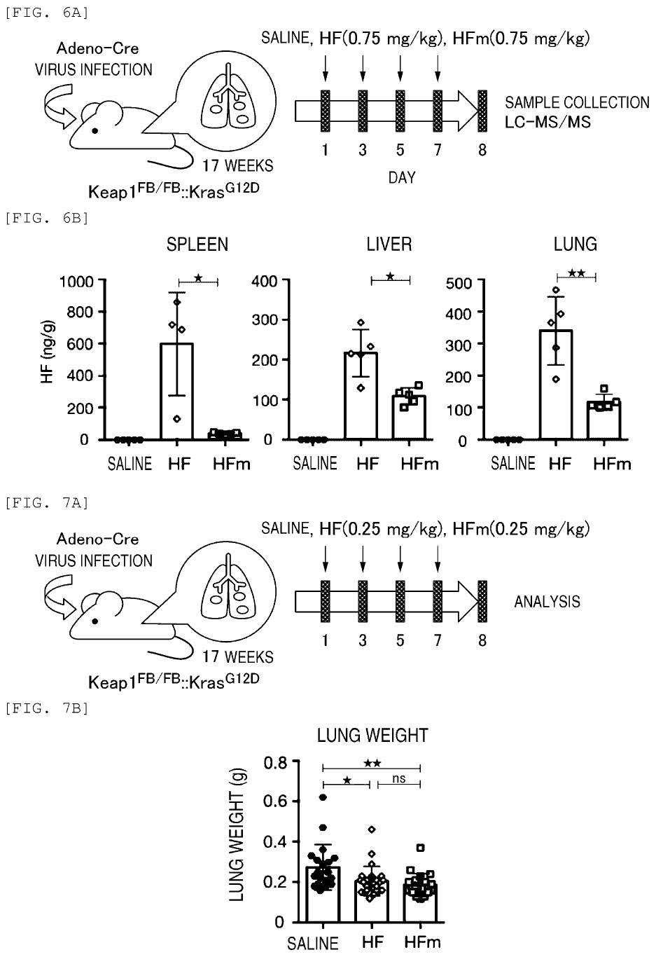

To examine the reason why HFm shows lower toxicity than HF, LC-MS/MS analysis was conducted again, and the HF concentrations of the tissues of cancer-bearing model mice were measured. From the mice which were treated with any of HF, HFm, and saline four times and sacrificed on the eighth day, three tissues, namely, the lungs, the spleen, and the liver, were obtained (FIG. 6A). Each tissue was homogenized together, and the existence of HF was observed by LC-MS/MS.

It was found that the HF level of the cancer-bearing lung was far lower in the mice treated with HFm than in the mice treated with free HF (FIG. 6B). At the end of the seven-day treatment protocol, the lung tumors of the HF- and HFm-treated mice were far smaller than the tumors of the mice evaluated before the treatment. Due to the EPR effect exhibited by the polymer micelles, HFm was not taken efficiently in the normal lung tissue (40.5 ng/g of the tissue weight). On the other hand, free HF in the blood was taken efficiently and highly accumulated in the normal lung tissue (598.4 ng/g of the tissue weight). The above results clearly show that HFm is accumulated preferentially in a tumor.

It was reproduced that the accumulated HF amounts in the spleen and the liver were lower with the administration of HFm than with the administration of free HF (FIG. 6B). It is believed that the significantly low accumulated HF amounts in the normal tissues after the HFm administration are behind the low systemic toxicity of HFm. Moreover, it was found that the administration of HFm causes a significantly higher level of HF accumulation in the spleen and the liver than in the lungs. The reason for the above difference is unclear so far. A likely explanation is that the EPR effect exhibited by polymer micelles functions strictly in the lung tissue. This seems to be an advantage of the use of HFm for lung cancer.

### Test Example 7: Low-Dose HF/HFm Therapy Suppresses Nrf2-Activated Tumor, but not as Effectively as High-Dose Therapy

From the above results, it was elucidated that, by the administration at the MTD, HFm has fewer side effects than free HF and exhibits an excellent effect on lung cancer. Thus, to examine whether the results would be reproducible when HF was administered to lung cancer at a lower dose than the MTD and whether HFm would have the similar effect to that of HF also at the basic effective dose (hereinafter also referred to as “BED”), HF and HFm treatment with administration at the BED, namely 0.25 mg/kg bw, was examined in lung cancer-bearing mice.

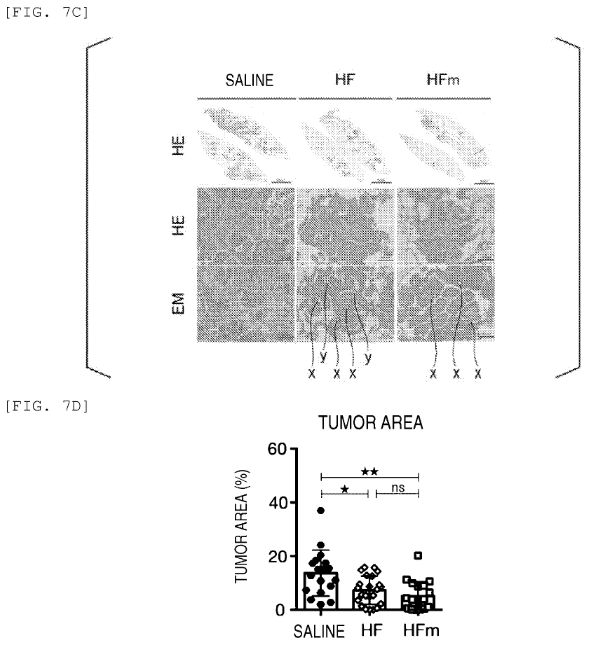

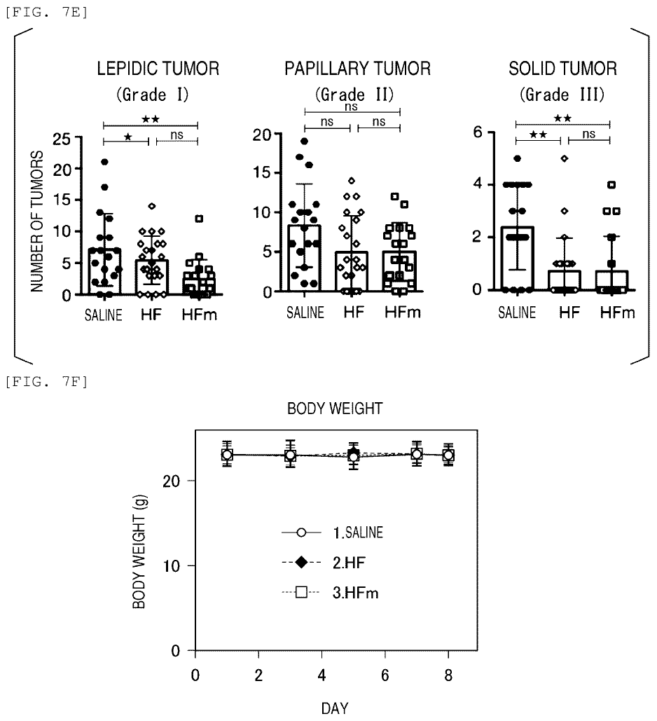

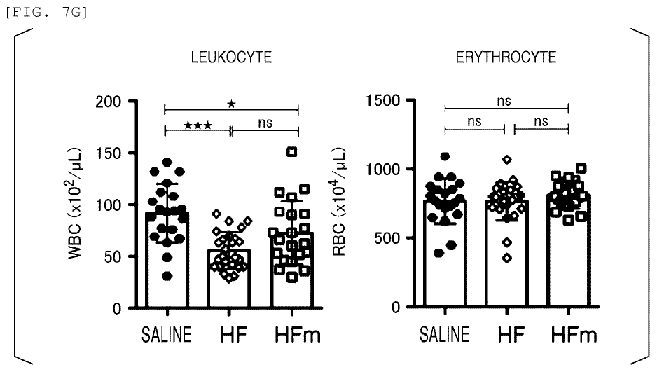

After KRAS-driven lung adenocarcinoma was grown for 17 weeks, the mice were treated with saline, HF (0.25 mg/kg), or HFm (0.25 mg/kg) every two days four times in total (FIG. 7A). In the same manner as in the high-dose (0.75 mg/kg) treatment, the HF and HFm treatment at the BED also effectively decreased the lung weight (FIG. 7B). When lung slices were stained with HE (FIG. 7C, top and middle), the tumor area was decreased significantly by the HF administration and the HFm administration also at the BED (FIG. 7D), but the tumor suppression effect was far weaker with the treatment at the low dose or the BED than with the treatment at the high dose or the MTD. While the tumor area decreased by 9.95% (saline vs. HF) and 15.77% (saline vs. HFm) at the high dose, the tumor area decreased only by 7.42% (saline vs. HF) and 9.49% (saline vs. HFm) at the low dose. Similarly, while the lung weight decreased by 37.6% in the HF-administered mice and 49.6% in the HFm-administered mice at the high dose, the lung weight decreased only by 25.7% (saline vs. HF) and 22.8% (saline vs. HFm) at the low dose (FIG. 4B and FIG. 7B).

Also with the 0.25 mg/kg bw treatment, the number of papillary lesions was far smaller with the HF or HFm administration than with the saline administration (FIGS. 7C, bottom and 7E). The numbers of solid tumors after the administration of 0.25 mg/kg bw of HF or HFm were smaller with significance than the number of solid tumors after the saline administration (FIG. 7E). However, contrary to the change observed with the high-dose therapy (FIG. 4E), the solid tumors did not disappear completely with the low-dose therapy, and the effectiveness of the HF treatment and that of the HFm treatment were equivalent. In all the mouse groups, the body weight changes due to the administration were equivalent (FIG. 7F), but the low-dose administration of HF decreased the leukocyte count with significance. The treatment with 0.25 mg/kg bw of HF did not affect the erythrocyte count. It is noticeable that the adverse influence was recovered when HFm was used (FIG. 7G). The above results show that the HF treatment at the BED causes severe toxicity on the leukocyte count. Important observation here is that the HFm treatment at the low dose exhibited the equivalent therapeutic effect to that of the treatment with the same amount of HF. Thus, the above results demonstrate that HFm acts as a safer and more potent therapeutic agent than HF and that HFm can be used for a therapy at a higher dose.

### Discussion

Abnormal activation of Nrf2 is frequently observed in various types of cancer and causes coordinate expression of various cytoprotective genes. Such overactivation of Nrf2 confers cancer cells with resistance to chemotherapies/radiotherapies and causes malignant transformation. Because no approved therapeutic method is available for treating such an Nrf2-activated cancer, finding a practicable therapeutic method for treating patients having such a tumor is an important unmet clinical need. HF has been identified as a potent Nrf2 inhibitor, and there are demonstrative evidences for its role as an anticancer chemical sensitizer by preclinical tests. However, in this test, it was also elucidated that administration of a large amount of HF to WT mice exhibits dose-dependent toxicity, such as significant decreases in the hematopoietic cells and the immune cells of the spleen, the thymus, and the bone marrow. Thus, to overcome the side effects, HFm, in which HF is encapsuled in micelles and which maintains the tumor suppression effect of HF but reduces the systemic toxicity thereof, was designed. When the effectiveness and the side effects of HFm were examined using the Keap1^{FB/FB}:Kras^{G12D} cancer-bearing model mice, it was elucidated that HFm reduces the tumor burden without causing immunosuppression or a decrease in hematopoietic cells. Moreover, it was found from the LC-MS/MS experiment that the relief of the side effects is caused because HFm slowly and continuously releases HF from the core. The results of this test support the idea that HFm is a suitable drug for treating an Nrf2-activated cancer through application of elaborated nanomedicine.

In this test, the immune cell groups and the hematopoietic cell groups were decreased significantly by the administration of HF. Although there are some publications which describe the toxicity of HF at a high dose, there is no publication which describes enhanced immunity and suppression of hematopoietic cells mediated by HF. It is speculated that there are two pathways, namely an Nrf2-dependent pathway and an Nrf2-independent pathway, as the pathways causing the toxicity of HF observed in this test. It was speculated that the Nrf2-independent pathway may be involved in immunosuppression promoted by the GCN2-eIF2 center. It has been reported that amino acid starvation response due to asparaginase promotes phosphorylation of eIF2 in a GCN2-dependent pattern and thus causes a decrease in the thymus and spleen sizes and depletion of B cells, CD4⁺ T cells, and CD8⁺ T cells. Because the suppression of Nrf2 by HF is driven by eIF2 phosphorylation through GCN2, the decreases in the immune and the hematopoietic cell populations may be caused by the same pathway.

Deletion of Nrf2 causes a decrease in hematopoietic stem cells and also causes a moderate decrease in precursor cells (c-Kit⁺) in the bone marrow. Thus, the decreases are speculated to be caused by an Nrf2-dependent pattern. It is noticeable that, in this test, while most of the examined cell groups were immune cells, moderate decreases in the erythrocytes, the hematocrit level, and the erythroblasts were also observed. The results of this test showed that the cell death by HF may widely occur and widely suggested the cellular HF toxicity. The toxicity of HF on lymphoid cells, such as CD4 single-positive cells, CD8 single-positive cells, and B cells, was more significant than the toxicity on bone marrow-derived macrophages and neutrophils. However, the mechanism of the phenomenon has not been elucidated. To treat an Nrf2-activated cancer and exhibit the effect of a chemotherapy at the maximum, Nrf2 inhibition using elaborated nanomedicine suited to the tumor is believed to be an excellent approach.

HF and HFm at the high dose completely destroyed solid tumors of Grade III in the Keap1^{FB/FB}:Kras^{G12D} cancer-bearing model mice. It was supposed that many tumors would develop in the lungs of the model mice, and it was speculated that Nrf2 inhibition would eradicate most Nrf2-activated cancers and that most of the remaining tumors would have Kras^{G12D} mutation only. It is known that a Kras-mutated tumor without Nrf2 activation is less invasive, and thus, there were no solid tumors in the lung slices of the mice treated with HF or HFm. Another explanation is the involvement of the metalloproteinase-2 (MMP-2) pathway because HF has been shown to suppress the expression of MMP-2. Because MMP-2 is involved in membrane invasion and metastasis of a tumor, it was speculated that HF acts to decrease the MMP-2 level and as a result eliminates solid tumors.

It is noticeable that HF is widely used in the veterinary field as antibiotics for animals and has been shown to have an anticancer action and an anti-metastasis property in various in vivo tests. It has been reported that HF inhibits the TGF-β signal pathway and suppresses progress of bone metastasis of melanoma, and it has been reported that suppression of MMP-2 expression suppresses metastasis of bladder cancer. HF has undergone various phase II human trials as a therapeutic agent for AIDS-related Kaposi's sarcoma and fibrotic diseases. Based on this test, the possibility that HFm has superior effectiveness and less side effects than other therapeutic methods targeting an Nrf2-activated tumor which is currently untreatable is proposed.

In summary, this test showed that HFm effectively removes an Nrf2-activated tumor in the lungs without exhibiting obvious systemic toxicity. The results of this test support the application of HFm to human tests because the clinical need for an Nrf2 inhibitor is high.

1. A conjugate comprising a block copolymer having a PEG chain block and a polypeptide chain block,
wherein one molecule or more of a compound (1) represented by the following general formula (1) is bonded to an amino acid side chain constituting the polypeptide chain block with a covalent bond through the oxygen atom of the hydroxy group of the compound (1): 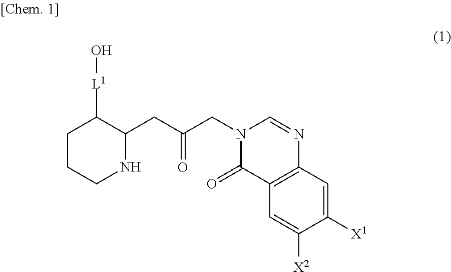
[wherein in the general formula (1), L¹ is a single bond or an alkylene group having one to five carbon atoms, wherein any methylene group in L¹ may be substituted with a carbonyl group or an ether bond as long as the oxygen atoms are not adjacent to each other, and X¹ and X² are each independently a hydrogen atom or a halogen atom.]

2. The conjugate according to claim 1, wherein the covalent bond is an ester bond or a urethane bond.

3. The conjugate according to claim 1, wherein one to 100 molecules of the compound (1) are bonded to one molecule of the block copolymer.

4. The conjugate according to claim 1, wherein the polypeptide chain block is a polyaspartic acid chain or a polyglutamic acid chain.

5. The conjugate according to claim 1, wherein the polymerization degree of the polypeptide chain block is 10 to 100.

6. The conjugate according to claim 1, wherein the polymerization degree of the PEG chain block is 50 to 900.

7. The conjugate according to claim 1, wherein a side chain of the polypeptide chain block forms a pharmaceutically acceptable salt.

8. The conjugate according to claim 1 which is represented by the following general formula (2): 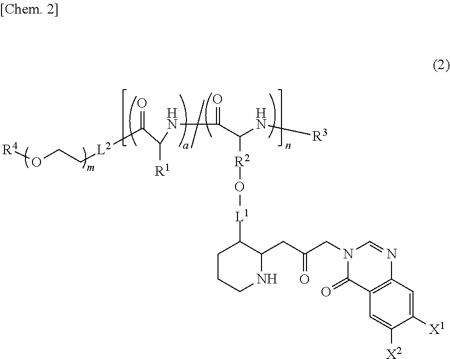
[wherein in the general formula (2), L¹ is a single bond or an alkylene group having one to five carbon atoms, wherein any methylene group in L¹ may be substituted with a carbonyl group or an ether bond as long as the oxygen atoms are not adjacent to each other, L² is a linking group, X¹ and X² are each independently a hydrogen atom or a halogen atom, m is an integer of 50 to 900 representing the polymerization degree of the PEG chain, n is an integer of 10 or more representing the polymerization degree of the polypeptide chain, a is a number of 0≤a<1 that represents the proportion of the constituent unit based on an amino acid to which the compound (1) is not bonded, b is a number of 0<b≤1 that represents the proportion of the constituent unit based on an amino acid to which the compound (1) is bonded, wherein a+b=1, R¹ is an amino acid side chain, R² is an amino acid side chain which forms the covalent bond with the compound (1), R³ is a hydrogen atom, a functional group, or an atomic group which constitutes the terminus of the polypeptide chain block, and R⁴ is a hydrogen atom, a functional group, or an atomic group which constitutes the terminus of the PEG chain block.]

9. The conjugate according to claim 1 which is represented by the following general formula (3): 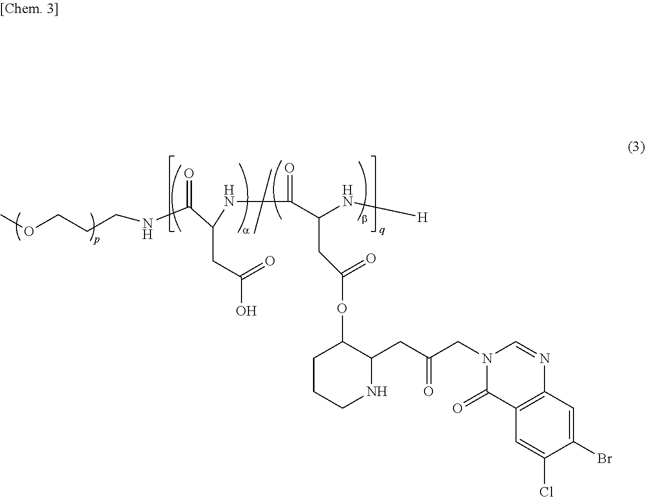
[wherein in the general formula (3), p is an integer of 170 to 380 representing the polymerization degree of the PEG chain, q is an integer of 25 to 55 representing the polymerization degree of the polypeptide chain, a is a number of 0.4≤α<0.7 that represents the proportion of the constituent unit based on aspartic acid to which halofuginone is not bonded, and β is a number of 0.3≤β≤0.6 that represents the proportion of the constituent unit based on aspartic acid to which halofuginone is bonded, wherein α+β=1.]

10. A micelle formed by association of a plurality of the conjugates according to claim 1.

11. The micelle according to claim 10 having a hydrodynamic diameter at 25° C. of 30 to 50 nm.

12. An anticancer agent comprising the conjugate according to claim 1 as an active ingredient.

13. An anticancer agent comprising the conjugate according to claim 2 as an active ingredient.

14. An anticancer agent comprising the conjugate according to claim 3 as an active ingredient.

15. An anticancer agent comprising the conjugate according to claim 4 as an active ingredient.

16. An anticancer agent comprising the conjugate according to claim 5 as an active ingredient.

17. An anticancer agent comprising the conjugate according to claim 6 as an active ingredient.

18. An anticancer agent comprising the conjugate according to claim 7 as an active ingredient.

19. An anticancer agent comprising the conjugate according to claim 8 as an active ingredient.

20. An anticancer agent comprising the conjugate according to claim 9 as an active ingredient.
